(19) United States
(12) Patent Application Publication
(10) Pub. No.: US 2025/0204956 A1
(43) Pub. Date: Jun. 26, 2025

(54) System And Methods For Providing Tamponade And Suction-Based Treatment To A Uterus

(71) Applicant: Stryker Corporation, Portage, MI (US)

(72) Inventors: Robert L. Esser, Rockford, MI (US); Jeffrey Brian Hensler, Portage, MI (US); Kathleen McClellan, Kalamazoo, MI (US); Lauren R. West, Cedar Park, TX (US); Lucas Wade, Austin, TX (US); Andrew Stanger, Portage, MI (US); Laura Frank, Greer, SC (US); Patrick Lafleche, Kalamazoo, MI (US); Joseph Michael O'Reilly, Kalamazoo, MI (US); Leif Norland, St. Joseph, MI (US); Spencer Gage Vansickle, Plainwell, MI (US); Denise Carmen Azcui, Portage, MI (US)

(73) Assignee: Stryker Corporation, Portage, MI (US)

(21) Appl. No.: 18/850,363

(22) PCT Filed: Mar. 24, 2023
(86) PCT No.: PCT/US2023/016283

§ 371 Date: Sep. 24, 2024

Related U.S. Application Data
(60) Provisional application No. 63/448,084, filed on Feb. 24, 2023.
(60) Provisional application No. 63/323,677, filed on Mar. 25, 2022.

(51) Int. Cl. A61B 17/42; A61B 17/00

(52) U.S. Cl. CPC A61B 17/42; A61B 2017/00557; A61B 2017/4216; A61B 2217/005

(57) ABSTRACT

Systems and methods for treating a uterus. The system provides for independent and/or simultaneous tamponade and suction-based treatments. A device includes an intrauterine portion coupled to or integrally formed with a cervical portion. A bladder may be disposed within an outer body and configured to receive fluid to be deployed. The outer body defines suction openings, and patient fluid may be suctioned through a volume defined between the bladder and the outer body. The device may include one or more spacing features or struts to maintain the volume. A distal cap may facilitate distal expansion for the intrauterine portion to engage the fundus of the uterus. A cervical portion may be deployed to an expanded configuration within the cervix. The system may include a conduit defining at least three fluid pathways, a connector, and a control hub configured to be coupled to the device or the conduit via a connector.

### PRIORITY CLAIM

This application claims priority to and all the benefits of U.S. Provisional Patent Application No. 63/448,084, filed Feb. 24, 2023, and U.S. Provisional Patent Application No. 63/323,677, filed Mar. 25, 2022, the entire contents of each being hereby incorporated by reference.

### BACKGROUND

Excessive bleeding during or following childbirth is a major cause of maternal morbidity. Postpartum hemorrhage (PPH) is an especially severe condition in which weak or absent contractions following childbirth fail to put sufficient pressure on bleeding vessels, a phenomena known as uterine atony.

It is known to treat PPH by deploying a balloon within the uterus to provide a tamponade. The pressure from the deployed balloon against the uterine wall facilities hemostasis. Separately, it has been shown that applying suction within the uterus may invoke uterine contractions, which itself often facilitates hemostasis. Owing to the fundamental differences in the aforementioned approaches, devices configured to provide tamponade are unable to provide for optimal suction, and devices configured to provide for suction are ill-designed to provide tamponade.

Therefore, there is a need in the art for a device providing for selectively providing one or both tamponade and suction-based treatments. There is a further need in the art for such a device that is also easily positionable within the uterus through the cervix or through a cesarian incision for intuitive deployment and operation.

### SUMMARY

A system for treating a uterus includes a device, and an optional control hub coupled to the device by a conduit. The device may be removably coupled to the control hub with a connector. The device provides for tamponade and suction-based treatments within the uterus, either independently or simultaneously, and each of varied and/or adjustable levels. The device includes an intrauterine portion, and a cervical portion coupled to the intrauterine portion. The cervical portion is optional.

The device includes a bladder coupled to the conduit. The conduit defines a suction pathway and an inflation pathway separate from the suction pathway. At least one suction port defined by the conduit is in fluid communication with the suction pathway. The bladder defines a first volume in fluid communication with the inflation pathway. The suction port is external to the bladder. The suction port may be disposed proximal to a position at which a base of the bladder is coupled to the conduit. Another one or more suction ports may be defined by the conduit at a position distal to the bladder.

The device may include an expandable outer body at least partially disposed over the bladder. A portion or an entirety of the bladder may be disposed within the outer body. A second volume is defined between the bladder and the outer body. The second volume and the first volume may not be in fluid communication with one another. The second volume is in fluid communication with the suction port of the conduit and the suction pathway. The outer body defines suction openings in fluid communication with the second volume. The outer body may define at least one recess with the suction openings being disposed within the recess. The suction openings may be of any suitable size or sizes and shape or shapes, and/or arranged any suitable spacing, positioning, and the like, and combinations thereof.

The second volume may be maintained by projections or other spacing features on an outer surface of the bladder, and/or on an inner surface of the outer body. The spacing features may be integrally formed with the bladder and/or the outer body, or discrete components. In certain implementations, the discrete components may be one or more struts. The struts may be positioned between the bladder and the outer body. The struts may be elongate and include projections defining a channel providing at least a portion of the second volume. The strut defines bores disposed within the channel. The bores of the strut are aligned with the suction openings of the outer body. There may be more or less than three struts, which may be oriented longitudinally, circumferentially, combinations thereof, or in other suitable configurations. A face of the strut is disposed opposite the projections and fixedly coupled to the inner surface of the outer body. In other variants, the strut may be fixedly coupled to the outer surface of the bladder, free-floating, fixedly coupled to another subcomponent of the device, or combinations thereof. The struts may include a flared portion near their respective distal ends that flare inwardly towards the conduit. The struts may be used in combination with the suction openings defined within recesses of the outer body.

The conduit defines an inflation port in fluid communication with the inflation pathway. The bladder may expand omnidirectionally, and the outer body may constrain the expansion of the bladder to provide a desired expansion profile to the intrauterine portion. Alternatively, the bladder may be formed from a flexible, non-resilient material that is furled or folded within the outer body. The outer body may stretch and otherwise conform to the shape of the bladder. Multiple bladders may be disposed within one or more outer bodies with each of the bladders configured to be independently and adjustably deployed to provide the desired expansion profile to the intrauterine portion.

The intrauterine portion may expand radially outward as well as longitudinally outward, i.e., axially distal. The device may include a distal cap movably disposed over a distal end portion of the conduit. The distal end portion may include a base fixedly coupled to a proximal portion of the conduit. The base may seal respective distal ends of the lumens or pathways of the conduit. The distal end portion may be tapered inwardly from the base. The distal cap defines a cavity sized to receive the distal end portion. A trunk of the distal cap may define the cavity, and a cap head may include a blunt tip. The bladder is coupled to the distal cap, and in particular a tip portion of the bladder may be coupled to the cap head of the distal cap. The outer body may not be directly coupled to the distal cap. The base of the bladder is fixedly coupled to the conduit, and the tip portion of the bladder is fixedly coupled to the distal cap. The distal cap is optional, for example, in implementations in which the bladder is formed from the flexible, non-resilient material.

In certain implementations, a collar may couple the intrauterine portion and the cervical portion. Alternatively, the intrauterine portion and the cervical portion may be formed as a single-piece component. In another variant, the collar may be formed with the intrauterine portion and the cervical portion as a single-piece component. The collar is coupled to the conduit and includes an inner hub, an outer hub, and a barrier coupling the inner hub and the outer hub. The barrier may extend radially outward from the inner hub to the outer hub. The inner hub may be proximal to the barrier, and the outer hub may define a cavity distal to the barrier. The cavity is in fluid communication with the second volume, and the suction port may be axially positioned within the cavity. A base of the outer body is coupled to the collar. The outer hub may include an outer flange, and the base of the outer body may be coupled to a portion of the outer hub that is distal to the outer flange. The base of the outer body may be coupled to the collar at a position that is proximal to where the bladder is coupled to the conduit. The base of the outer body is spaced apart from the conduit.

The collar may define suction openings. The suction openings may extend through the outer hub, and be positioned within the outer flange. The suction openings are in fluid communication with the cavity, and further in fluid communication with the second volume. Indicia may be disposed on or coupled to the collar to provide visual guidance to the user during insertion of the device. The indicia may be a colored marker, such as colored material on the outer flange.

The cervical portion may include a seal body. The seal body may include the head, and a base. The head may be coupled to the collar, and the base may be coupled to the conduit. The head may be coupled to the outer hub of the collar at a position that is proximal to the flange. The seal body may be of variable thickness along its length that is defined between the head and the base. The axial location of the variable thicknesses may be designed based on a desired expansion profile. The seal body may define a third volume in fluid communication with a second inflation pathway defined by the conduit. The conduit further defines a second inflation port providing fluid communication between the second inflation pathway and the third volume. The barrier of the collar may maintain fluid separation between the second volume and the third volume. The cervical portion may include another bladder to force expansion of the seal body to the expanded configuration.

The system may include the connector to facilitate removable coupling sections of the conduit. The connector is optional. The conduit defines the suction pathway, the first inflation pathway, and the second inflation pathway, and thus each of the sections of the conduit defines portions of the pathways when removably coupled together via the connector. The lumens of the conduit may be of different sizes. The effective cross-sectional area of the suction pathway may be greater than an effective cross-sectional area of each of the first and second inflation pathways. The conduit may have a constant outer diameter. The relative cross-sectional areas may be such that the effective cross-sectional area of the suction pathway is within the range of 0.50 to 0.65 square inches.

The control hub includes a module housing coupled to a vacuum line, a first supply line, and a second supply line. The control hub selectively and individually controls deployment of the intrauterine portion, deployment of the cervical portion, and vacuum being drawn through the device. The control hub includes at least a first user input, and optionally a second user input. The first user input receives an input to actuate a vacuum valve to permit or prevent the vacuum being drawn on the suction openings and the second volume. The second user input receives another input to actuate a first inflation valve to permit or prevent fluid passing through the first supply line to within the bladder of the intrauterine portion. The vacuum valve is configured to be arranged in fluid communication with the suction pathway of the conduit, and the first inflation valve configured to be arranged in fluid communication with the first inflation pathway of the conduit. The first and second user inputs may be switches, knobs, or electronically-actuated buttons, or the like. The control hub may include a second inflation valve arranged in fluid communication with the second inflation pathway of the conduit. The second inflation valve is operable to permit or prevent fluid from the second fluid source to be directed to or removed from the second volume. The first inflation valve and/or the second inflation valve may be a self-sealing fitting, for example, a self-sealing Luer fitting configured to be coupled with a syringe of saline.

In certain implementations, the device may include at least one sensor arranged in wired or wireless communication with a controller. The sensor may be disposed on the intrauterine portion, or the cervical portion, or both. The sensor is configured to generate and transmit a signal to the controller to determine a force being applied to the uterine wall by the device. The sensors may be contact sensors, force sensors, displacement sensors, or the like. The controller may be configured to operate a pump based on the signals from the sensors to accommodate the forces on the intrauterine portion from the uterine wall. The pump may be an electronically-actuated syringe, or another suitable pressure-producing device. The contact sensors also may be configured to transmit a contact signal to a controller or an output device with the signal indicative that the head is contacting a uterine wall of the uterus. The sensors, the controller, and the pump are optional and the characteristics of the operation of the device may be obtained through other means, such as electronic sensing and feedback of the medical waste collection system.

Therefore, according to certain aspects of the present disclosure, a device for treating a uterus is provided. The device includes a conduit defining a suction pathway and an inflation pathway. A bladder is coupled to the conduit and defining a first volume in fluid communication with the inflation pathway. The device includes an expandable outer body at least partially disposed over the bladder and defining suction ports. A second volume defined between the bladder and the outer body is in fluid communication with the suction ports of the outer body and the suction pathway of the conduit. The conduit defines a suction port external to the bladder with the suction port in fluid communication with the suction pathway.

An entirety of the bladder may be disposed within the outer body. The first volume and the second volume may not be in fluid communication. A channel may be defined to maintain separation between the bladder and the outer body with the bladder in an inflated state for bodily fluids to be drawn through the second volume and into the suction pathway. The channel may be defined by spacing features coupled to or integrally formed with the outer body and/or the bladder. The spacing features may be a strut coupled to the outer body and/or the bladder. The strut or the spacing features defines the channel, and bores aligned with the suction ports of the outer body. The bores may be disposed within the channel. The strut(s) may be oriented longitudinally along an inner surface of the outer body.

The conduit may define a suction port positioned proximal to the bladder, and another suction port position distal to the bladder. The conduit may define an inflation port positioned distal to the suction port and distal to a base of the bladder. The bladder may be formed from a resilient material configured to expand with inflation fluid being directed through the inflation pathway and into the first volume, wherein the outer body is formed from a resilient material configured to conform to the expansion of the bladder. Alternatively, the bladder may be formed from a flexible, non-resilient material configured to unfurl with inflation fluid being directed through the inflation pathway and into the first volume, wherein the outer body is formed from a resilient material configured to conform to a shape of the bladder.

The bladder may include a base coupled to the conduit, and wherein the bladder is not otherwise coupled to the conduit. Alternatively, the conduit may include a distal end portion, and the device further includes a distal cap movably disposed over the distal end portion, and wherein the bladder is coupled to the distal cap. The distal end portion may be tapered with the distal cap including a blunt tip. The distal cap may include a trunk, and a cap head extending distally from the trunk. The cap head may have an outer diameter less than an outer diameter of the trunk with a distal tip portion of the bladder is coupled to the cap head. The distal cap may define a cavity sized to receive the distal end portion of the conduit. The outer body may not be directly connected to the distal cap.

The conduit may define a second inflation pathway. The suction pathway and the first and second inflation pathways may be partitioned channels within the conduit. The conduit may have a constant outer diameter and wherein an effective cross-sectional area of the suction pathway is greater than an effective cross-sectional area of each of the first and second inflation pathways. The device may further include an expandable cervical seal coupled to the conduit and defining a third volume in fluid communication with the second inflation pathway. The cervical seal may be formed from a sidewall comprising an outer surface opposite an inner surface to define a thickness therebetween. The thickness of the sidewall may vary between opposing ends of the cervical seal.

A collar may be coupled to the conduit with the outer body and fixedly coupled to the collar, wherein the collar comprises a barrier arranged to maintain fluid separation between the second volume and the third volume. The collar and the cervical seal may be integrally formed, or be separate components fixedly coupled to one another. The conduit may further define a second inflation port positioned proximal to the barrier. The collar may define additional suction ports in fluid communication with the second volume and the suction pathway.

### BRIEF DESCRIPTION OF THE DRAWINGS

Advantages of the inventions will be readily appreciated with the written description being considered in connection with the following drawings.

FIG. 1 is a representation of a system in accordance with the implementations of the present disclosure with an intrauterine device of the system being positioned within female anatomy.

FIG. 2 is a perspective view of the system.

FIG. 3 is a perspective view of the device.

FIG. 4A is an exploded view of a first variant of the device.

FIG. 4B is an exploded view of a second variant of the device.

FIG. 5A is a plan sectional view of the device of FIG. 3 taken along line 5-5, and representative of the first variant.

FIG. 5B is a plan sectional view of the device of FIG. 3 taken along line 5-5, and representative of the second variant.

FIG. 6A is an elevation sectional view of the intrauterine device of FIG. 3 taken along line 6-6, and representative of the first variant. Each of an intrauterine portion and a cervical portion of the device are shown as deployed in an expanded configuration.

FIG. 6B is an elevation sectional view of the intrauterine device of FIG. 3 taken along line 6-6, and representative of the second variant. Each of an intrauterine portion and a cervical portion of the device are shown as deployed in an expanded configuration.

FIG. 7 is a perspective view of a strut of the device.

FIG. 8 is a perspective view of a collar of the device.

FIG. 9 is an elevation view of another implementation of the device. The intrauterine portion and the cervical portion are shown as undeployed in a collapsed or insertion configuration.

FIG. 10 is an elevation view of the device of FIG. 9 with the intrauterine portion and the cervical portion shown as deployed in the expanded configuration.

FIG. 11 is an elevation view of the device of FIG. 9 with the intrauterine portion shown as undeployed and the cervical portion shown as deployed.

FIG. 12 is an elevation view of the device of FIG. 9 with the intrauterine portion shown as deployed and the cervical portion shown as undeployed.

FIG. 13 is a front or first perspective view of a connector removably coupling two portions of a conduit of the system. Lumens of the first portion of the conduit are shown in section.

FIG. 14 is a rear or second perspective view of the connector and the conduit. Lumens of the second portion of the conduit are shown in section.

FIG. 15 is a perspective view of a female component of the connector.

FIG. 16 is a perspective view of a male component of the connector.

FIG. 17 is an elevation sectional view of the connector and the conduit of FIG. 14 taken along lines 17-17.

FIG. 18 is an exploded view of a control hub.

FIGS. 19A-19D are alternative implementations of the intrauterine portion of the device.

FIG. 20A-20D are implementations of the cervical portion of the device.

FIG. 21 is an elevation view of another implementation of the system in which at least one sensor of the device is configured to generate signals indicative of an operational state of the intrauterine portion when deployed. A controller is configured to operate a pump based on the signals received from the sensor.

### DETAILED DESCRIPTION

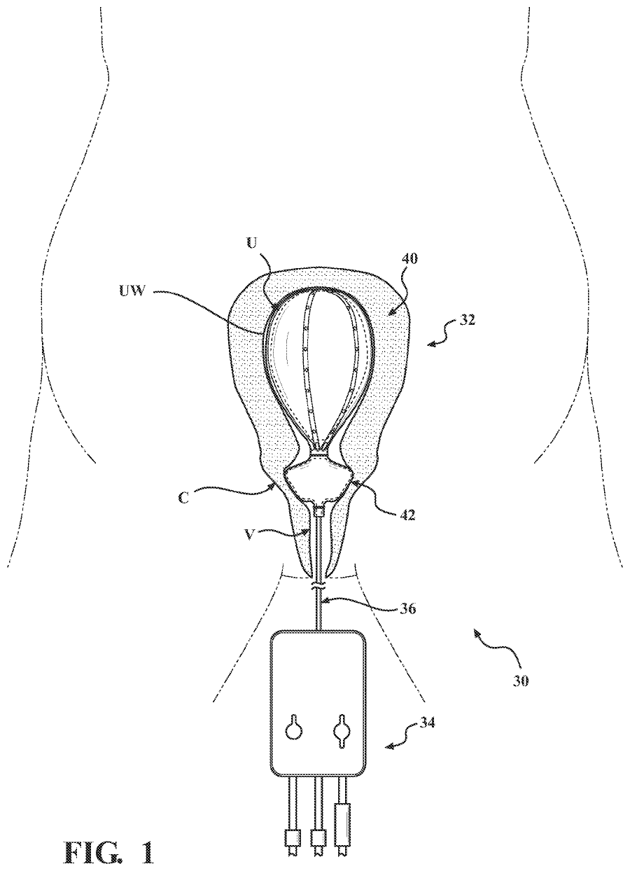

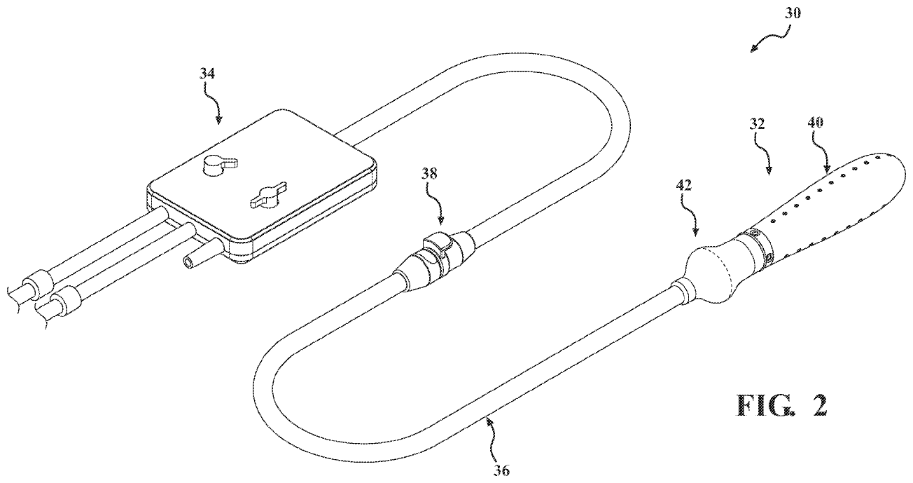

FIGS. 1 and 2 depict a system 30 for treating a uterus, most notably, occurrences of postpartum hemorrhage. The system 30 includes a device 32 configured to be positioned and deployed within the female anatomy, and a control hub 34 coupled to the device 32 by a conduit 36. The device 32 may be removably coupled to the control hub 34 with a connector 38. The control hub 34 is configured to be arranged in fluid communication with a vacuum source, a first fluid source, and/or a second fluid source. One suitable vacuum source is integrated on a medical waste collection system sold under the tradename Neptune by Stryker Corporation (Kalamazoo, Mich.), and disclosed in commonly-owned U.S. Pat. No. 7,621,898, issued Nov. 24, 2009, and U.S. Pat. No. 11,160,909, issued Nov. 2, 2021, the entire contents of each being hereby incorporated by reference. The first fluid source and/or the second fluid source may be a reservoir of saline or other suitable liquid. The reservoir may be a preloaded syringe. Alternatively, it is contemplated that the conduit 36 may be coupled to the vacuum source and/or the fluid sources directly or through indirectly another intermediate component. In other words, the control hub 34 may be optional in certain implementations.

The device 32 provides for tamponade and suction-based treatments within the uterus, either independently or simultaneously. Referring to FIG. 1, the device 32 includes an intrauterine portion 40 configured to be positioned within the uterus (U). The device 32 may include a cervical portion 42 coupled to the intrauterine portion 40 and configured to be positioned within or adjacent to the cervix (C), and more particularly the external cervical os. The cervical portion 42 may be deployed to seal the cervix to maintain a position of the device 32 within the female anatomy, and maximize suction being drawn from within the uterus from the vacuum source. With the device 32 supported in situ, the intrauterine portion 40 may be deployed to an expanded configuration within the uterus. The deployment of the intrauterine portion 40, as well as a shape of the intrauterine portion 40 itself, facilitate the tamponade-based treatment by engaging the uterine wall (UW), including that of the fundus. The vacuum-based treatment may be invoked with or without deployment of the intrauterine portion 40 to the expanded configuration. Thus, the device 32 of the present disclosure may advantageously be “dual-mode” to treat postpartum hemorrhage by facilitating homeostasis with the tamponade-based treatment, and evacuating excessing bleeding within the uterus with the suction-based treatment. Further, the shape and mechanism of deployment of the intrauterine portion 40 permits the device 32 to accommodate a range of shapes and sizes of uteri, and a range of stages of postpartum hemorrhage. It is further contemplated that the device 32 may be used only for drainage-based treatment in which no suction is drawn through the device 32, for drainage- or suction-based treatments in which no tamponade is provided, or for tamponade-based treatments in which no suction is drawn through the device 32.

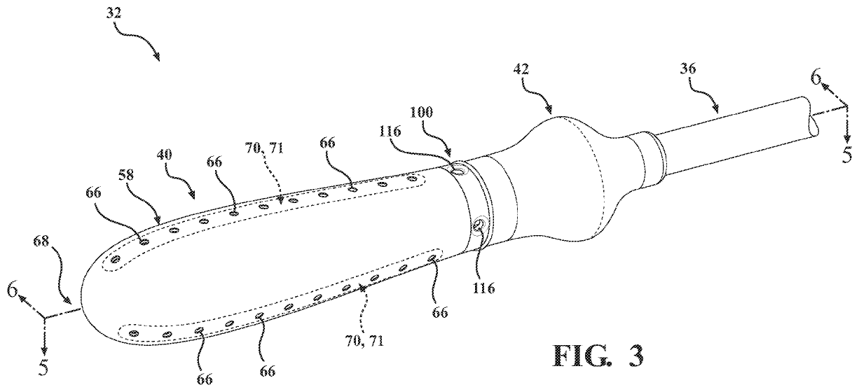

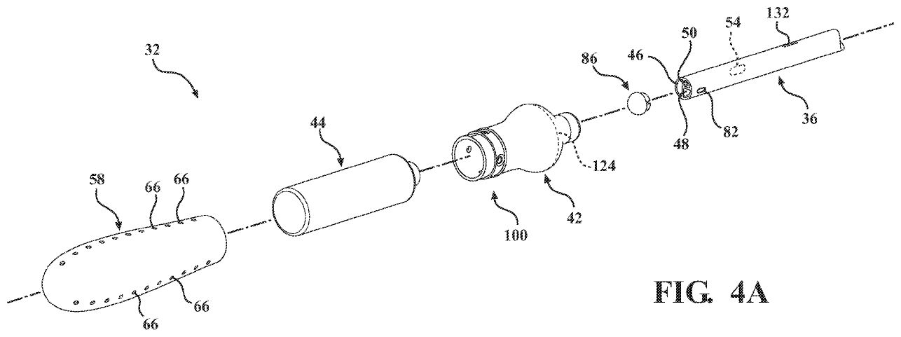

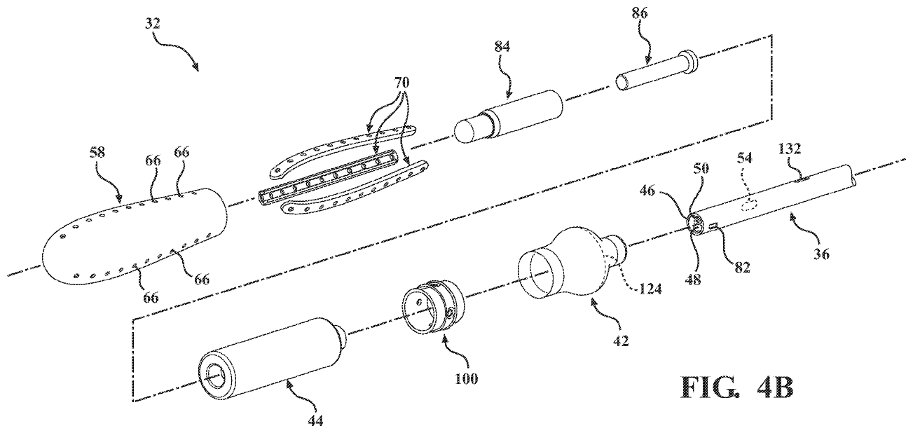

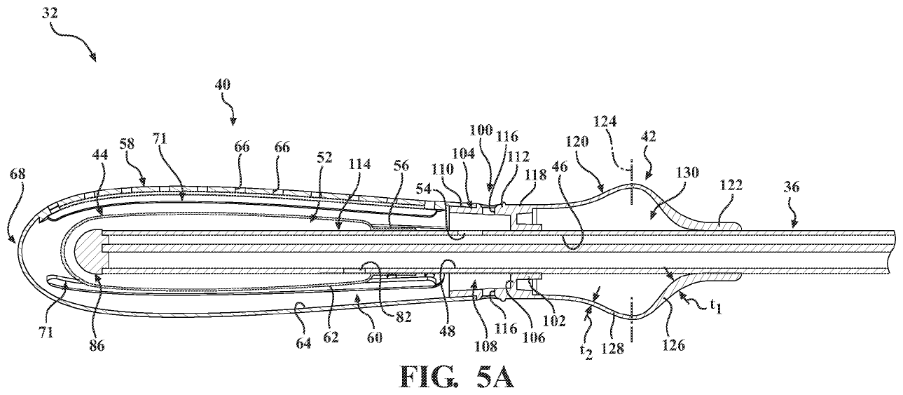

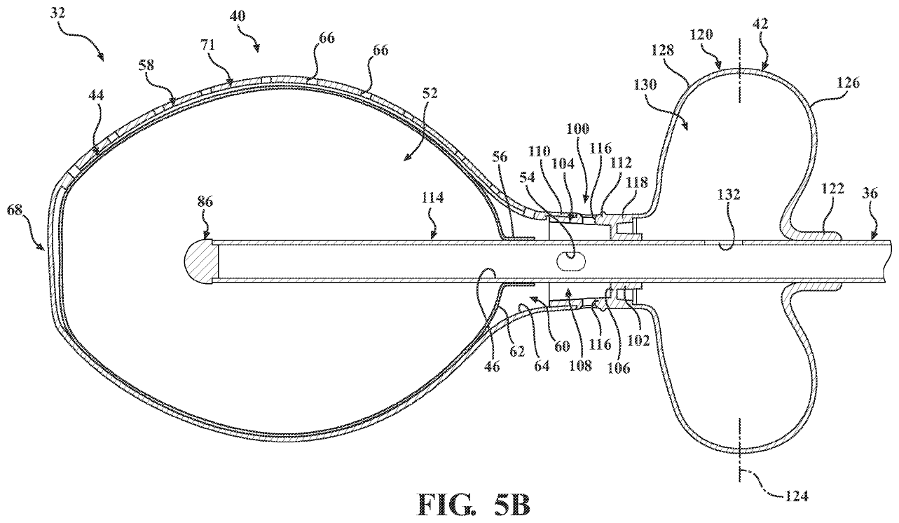

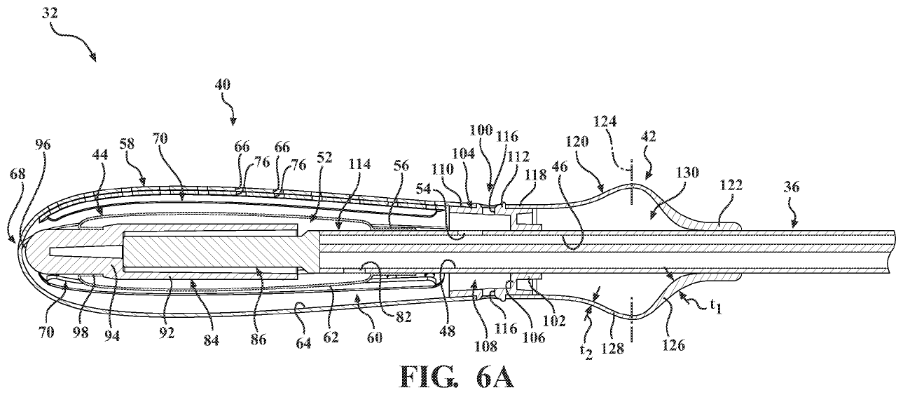

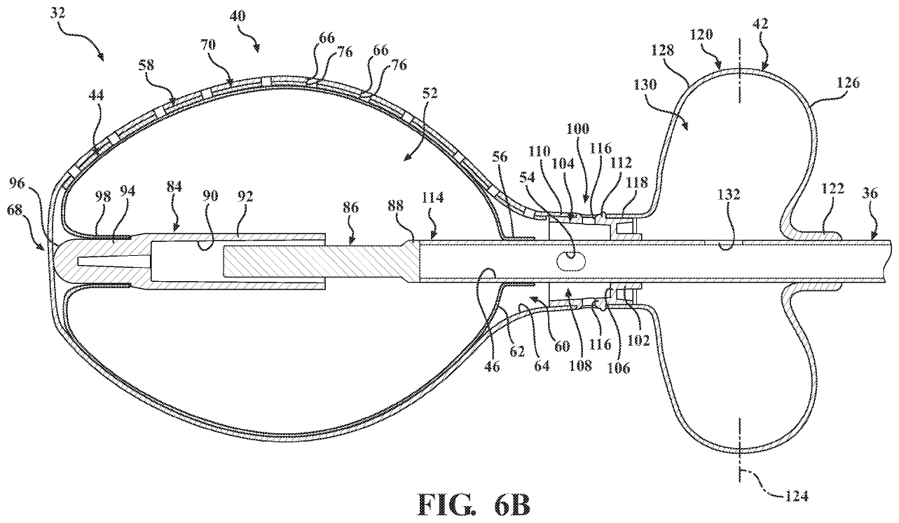

Referring to FIGS. 3, 4A and 4B, the device 32 includes the conduit 36, and a bladder 44 coupled to the conduit 36. The conduit 36 defines a suction pathway 46 and an inflation pathway 48 separate from the suction pathway. In the illustrated implementation, the conduit 36 is a multi-lumened tube with internal barriers separating the respective lumens to define the suction pathway 46 and the inflation pathway 48. Alternatively, the conduit 36 may be a multi-tube bundle with the respective tubes defining the suction pathway 46 and the inflation pathway 48. The conduit 36 need not be of continuous length, but rather formed from fixedly or removably coupled segments or sections. A suction port 54 defined by the conduit 36 is in fluid communication with the suction pathway 46 (see also FIGS. 5A-6B). The suction pathway 46 is configured to be arranged in fluid communication with the suction source, and the inflation pathway 48 is configured to be arranged in fluid communication with the first fluid source.

The bladder 44 defines a first volume 52 in fluid communication with the inflation pathway 48, and the suction port 54 is external to the bladder 44. The suction port 54 may be disposed proximal to a position at which a base 56 of the bladder 44 is coupled to the conduit 36. Additionally or alternatively, additional suction ports may be defined by the conduit proximal or distal to the bladder 44. Therefore, according to one implementation of the device 32, the bladder 44 of the intrauterine portion 40 is configured to be deployed to the expanded configuration by directing or pumping fluid from the first fluid source through the inflation pathway 48 and into the first volume 52. The extent to which the bladder 44 may be expanded is settable or selectable by the user and based on the clinical indications, and/or controlled through other means disclosed herein. As used herein, the expanded configuration means any widening or lengthening of the shape of the bladder 44 beyond its natural or default state, and therefore includes partial expansion as well as a maximum expansion permitted by the properties of the bladder 44. The expanded configuration may or may not result in the intrauterine portion 40 engaging the uterine wall. With the bladder 44 in the expanded configuration, patient fluid (e.g., blood associated with the PPH) may be drawn through the suction port 54 and into the suction pathway to be collected in a waste receptacle, such as the on-board container of the Neptune system. The bodily fluids may be collected and analyzed to determine concentration of blood within the fluid. For example, a device (not shown) may be positioned in-line with the conduit 36 and include sensors configured to detect characteristics of the fluid passing therethrough. The sensors may be optical light sensors, optical camera sensors, ultrasonic sensors, infrared sensors, volumetric flow sensors, weight sensors, or the like.

The device 32 may include an expandable outer body 58 at least partially disposed over the bladder 44. As best shown in FIGS. 5A-6B, an entirety of the bladder 44 is disposed within the outer body 58, and a second volume 60 is defined between the bladder 44 and the outer body 58. In other words, the second volume 60 may be any void(s) bounded by an outer surface 62 of the bladder 44 and an inner surface 64 of the outer body 58. The second volume 60 is in fluid communication with the suction port 54 of the conduit 36, and therefore also in fluid communication with the suction pathway 46.

The outer body 58 defines suction openings 66 in fluid communication with the second volume 60. The suction openings 66 are configured to be exposed to the anatomical environment within the uterus. The suction drawn on the device 32 from the vacuum source is configured to draw the bodily fluids through the suction openings 66, the second volume 60, and through the suction port 54 to within the suction pathway 46 of the conduit 36. The size, shape, quantity, positioning, spacing, and the like, of the suction openings 66 may be designed to impart a desired suction profile within the uterus based on, among other considerations, anticipated areas of fluid pooling as well as desired fluid flow rates through the device 32 at given levels of suction. In the illustrated implementation, the suction openings 66 are arranged in columns oriented longitudinally along the outer body 58. The columns are radially disposed about the outer body 58, and more particularly equiangularly spaced radially about the outer body 58. Within each of the columns, the suction openings 66 are equally spaced axially. Even more specifically, the illustrated implementation includes three columns each with eleven of the suction openings 66 with all of the suction openings 66 being of the same size. The columns advantageously provide for suction along nearly an entirety of the length of the intrauterine portion 40, and are also arranged near a distal end 68 of the device 32 to be positioned near the fundus.

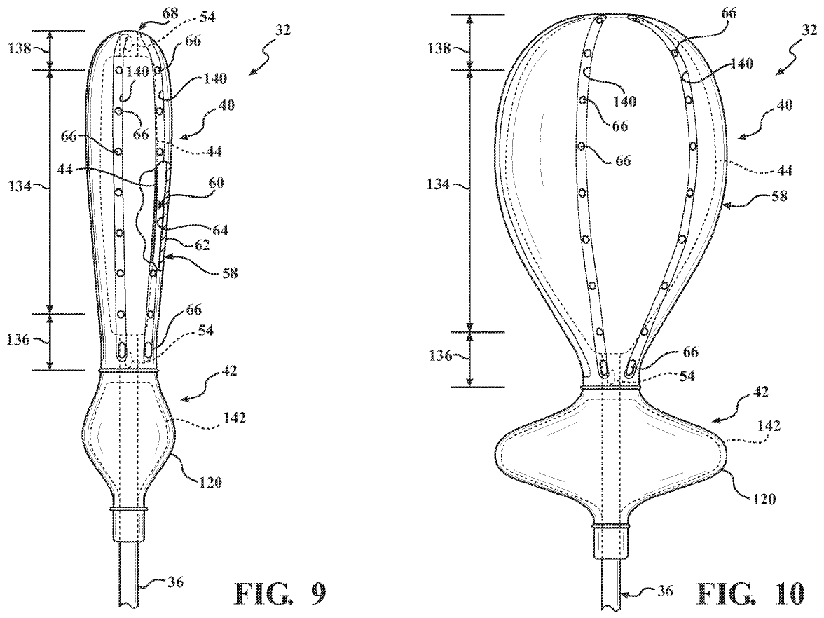

Numerous alternatives are contemplated to the size, shape, quantity, spacing, positioning, and combinations thereof, of the suction openings 66. The suction openings 66 may be of the same or different size. For example, one or more of the suction openings 66 nearer the cervical portion 42 may be larger than the suction openings 66 nearer the distal end 68 of the device 32 (see FIGS. 9-12). Alternatively, one or more of the suction openings 66 nearer the distal end 68 may be larger than the suction openings nearer the cervical portion 42. The diameter of the suction openings 66 may be between one and ten millimeters, and more particularly between one and five millimeters. The shape of the suction openings 66 need not be circular, and may assume otherwise suitable geometries, such as elongate or slit-like, elliptical, oblong, or the like. The quantity of the suction openings 66 may be varied, for example, each column including more or less than eleven openings arranged in more or less than three columns. More or less of the suction openings 66 may be concentrated nearer to the cervical portion 42 than nearer to the distal end 68, or nearer to the distal end 68 than nearer to the cervical portion 42. Along those lines, the spacing of the suction openings 66, within the columns or in general, may also be varied. The positioning of the suction openings 66 may be in a random, a partially uniform, or a uniform arrangement. The uniform arrangement may include the columns, or may be “rows” such as being circumferentially-spaced in a ring-like arrangement with the rings spaced apart axially from one another. The uniform arrangement may be zig-zagged, serpentine, or helical. The system 30 may be packaged in a kit including versions of the device 32 each having differential characteristics of the suction openings 66, selection of which may be based on the clinical indications or other considerations.

The second volume 60 and the first volume 52 are not in fluid communication with one another. In other words, the bladder 44 is formed from a fluid impermeable material configured to prevent ingress or egress of fluid into or from within the bladder 44, respectively. As a result, the tamponade and suction-based treatments may be effectuated either independently or simultaneously, and a magnitude of expansion and a vacuum level may be independently or codependently controlled. Such functionality advantageously addresses varied clinical scenarios requiring, for example, greater tamponade-based treatment with lesser suction-based treatment, or greater suction-based treatment with lesser tamponade-based treatment. The magnitude of expansion and the vacuum level may be individually set via user input on the control hub 34 to be described, or another input device.

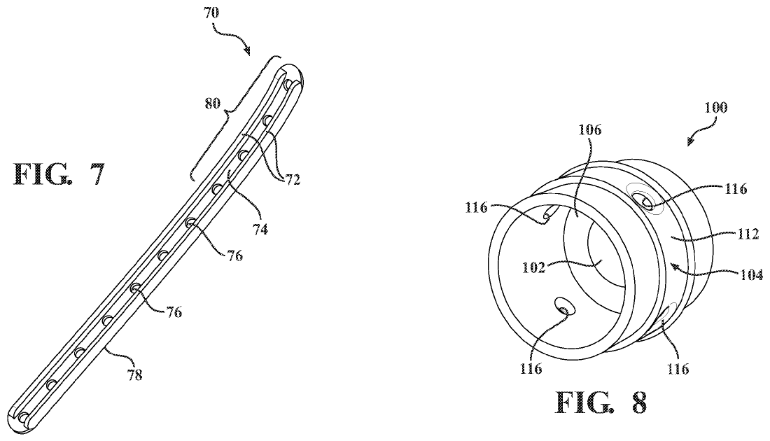

To facilitate a suction or flow path between the bladder 44 and the outer body 58 (i.e., the second volume 60), the device 32 may include one or more struts 70 or spacing features 71. The struts 70 or the spacing features 71 are configured to maintain separation between the bladder 44 and the outer body 58 with intrauterine portion 40 in the expanded configuration for the bodily fluids to be drawn through the second volume 60 and into the suction pathway 46. Referring to FIG. 7, the strut 70 may be elongate and include projections 72 defining a channel 74 The strut 70 defines bores 76 disposed within the channel 74. A face 78 of the strut 70 is disposed opposite the projections 72. The spacing features 71 may be shaped akin to the strut 70 illustrated in FIG. 7, but otherwise be integrally formed with the forming of the outer body 58. In other words, the inner surface 64 of the outer body 58 may be formed through a suitable manufacturing techniques to include the projections 72 defining the channel 74, wherein the projections 72 are the spacing features 71. FIGS. 4A, 5A and 5B depict a first variant of the device 32 in which the spacing features 71 are provided.

FIGS. 4B, 6A and 6B depict a second variant of the device 32 in which the struts 70 are separate components coupled to the outer body 58. In particular, the strut 70 is positioned between the bladder 44 and the outer body 58. The illustrated implementation shows the face 78 engaging the inner surface 64 of the outer body 58, and the projections 72 engaging the outer surface 62 of the bladder 44. The bores 76 of the strut 70 are aligned with the suction openings 66 of the outer body 58. In such an arrangement, the channel 74 of the strut 70 may be considered to define most the second volume 60 with intrauterine portion 40 in the expanded configuration. In other words, the bladder 44 may be formed from resiliently flexible material to generally conform to the outer body 58 and about the strut 70, but the channel 74 of the strut 70 is sufficiently narrow such that the outer surface 62 of the bladder 44 does not extend therein. During administration of the suction-based treatment, the bodily fluids are drawn through the suction openings 66 of the outer body 58 and the bores 76 of the strut 70. The bodily fluids travel proximally within the channel 74 to be drawn through the suction port 54 and into the suction pathway 46 of the conduit 36. The strut 70 (and/or the spacing features 71) facilitate suction along nearly the entirety of the length of the intrauterine portion 40 while still permitting for significant expansion of the intrauterine portion 40 within the uterus in a manner to be described. Known devices with a suction opening at a distal end of the device fail to adequately address such considerations. In the implementation

The struts 70 may be fixedly coupled to the inner surface 64 of the outer body 58, for example, with adhesive or an overmolding operation. Additionally or alternatively, the struts 70 may be fixedly coupled to the outer surface 62 of the bladder 44. In still another variant, the struts 70 may be free-floating, or fixedly coupled to another subcomponent of the device 32. The projections 72 may be ridges or rails, as shown, or other suitable spacing features such as nubs, fingers, contours, or the like. shown in FIG. 4B, there are three struts 70, hereinafter addressed in the plural. The struts 70 or the spacing features 71 may be oriented longitudinally and radially spaced within the outer body 58 and about the conduit 36. More particularly, the struts 70 the spacing features 71 may be equiangularly spaced radially within the outer body 58 and about the conduit 36.

The struts 70 may be formed from flexible materials so as to deform to be a contoured shape of the intrauterine portion 40 in the expanded configuration. In other words, the struts 70 may be elongate and flexible to permit bending of the struts 70 along their lengths. The struts 70 may include a flared portion 80 near their respective distal ends. The flared portions 80 of the struts 70 collectively flare inwardly towards the conduit 36. Owing to the bladder 44 and the outer body 58 being generally bulbous with the intrauterine portion 40 in the expanded configuration, the flared portion 80 facilitates the struts 70 accommodating a larger radius of curvature near a distal end of the bulbous shape. The flared portion 80 also facilitates improved insertion of the intrauterine portion 40 through the cervix and into within the uterus in manners to be further described. In the first variant including the spacing features 71 integrally formed with the outer body 58, the flexible material from which the outer body 58 is formed imparts similar flexibility to the spacing features 71 (e.g., the projections 72).

Like the suction openings 66 of the outer body 58 to which the bores 76 of the struts 70 are aligned, numerous alternatives are contemplated to the size, shape, quantity, spacing, positioning, and combinations thereof, of the struts 70. For examples, the struts 70 may be in a ring-like or a cage-like arrangement, or may be zig-zagged, serpentine, or helical. It is further contemplated that the struts 70 may be discrete subcomponents, as shown, or integrally formed with at least one of the bladder 44 and the outer body 58. The latter may include the projections 72 being formed with the outer body 58 through a molding or other suitable manufacturing processes. The projections 72 may be nubs, fingers, ridges, or contours, or of other suitable geometries. Other means for providing separation between two expandable bodies for a flow path therebetween are considered contemplated within the present disclosure. Additionally or alternatively, and in particular in implementations in which there is no outer body 58, the projections or additional projections may be disposed on an outer surface of the bladder 44 to maintain separation between the bladder 44 and the uterine wall.

Deployment of the intrauterine portion 40 is described with reference to FIGS. 5A-6B. The conduit 36 defines an inflation port 82 in fluid communication with the inflation pathway 48. The first volume 52 is configured to receive fluid under pressure to cause the bladder 44 to expand. The bladder 44 may be configured to expand omnidirectionally, and the outer body 58 may constrain the expansion of the bladder 44 to provide a desired expansion profile to the intrauterine portion 40. In other words, the bladder 44 is formed from a resilient material configured to expand with inflation fluid being directed through the inflation pathway 48 and into the first volume 52, and the outer body 58 is formed from a resilient material configured to conform to the expansion of the bladder 44. In particular, the dimensions (e.g., a wall thickness, default shape, etc.) and the materials of the outer body 58 may be selected accordingly. One example includes the outer body 58 being formed from silicone. Furthermore, the struts 70 or the spacing features 71 being on the outer body 58 may further impart the desired expansion profile by limiting an extent to which the outer body 58 may expand (e.g., stretch). Suitable materials for the intrauterine portion 40 and/or the cervical portion 42 may include polymers such as elastomers, nylons, resins, thermoplastics, thermosets, or the like, and exemplary manufacturing techniques include injection molding, blow molding, three-dimensional printing, among others. The materials may have an elastic moduli with the range of 0.01 to 2.0 Gigapascals (GPa), and more particularly within the range of 0.1 to 1.0 GPa.

In another implementation, the bladder 44 may be designed to provide the contoured profile with the outer body 58 conforming the shape of the bladder 44 with little resistance. In other words, the bladder 44 may be formed from a flexible, non-resilient material. Since the bladder 44 is non-resilient and therefore does not stretch more than minimally, the first volume 52 is predetermined or fixed and needs to be accommodated within the outer body 58. To that end, the bladder 44 may be furled or folded, and configured to unfurl or unfold with inflation fluid being directed through the inflation pathway 48 and into the first volume 52. The outer body 58 may be formed from the resilient material configured to stretch and otherwise conform to the shape of the bladder 44. Additionally or alternatively, it is further contemplated that multiple bladders may be disposed within one or more outer bodies with each of the multiple bladders configured to be independently and adjustably deployed to provide the desired contoured profile to the intrauterine portion 40.

FIGS. 4A-6B show the inflation port 82 being distal to the suction port 54 and distal to the base 56 of the bladder 44 (i.e., the position at which the bladder 44 is coupled to the conduit 36). As a result, the infusion fluid may be directed into the first volume 52 as desired without compromising the bodily fluids being drawn through the second volume 60 under suction. The intrauterine portion 40 is configured to expand radially outward, for example, to engage the uterine wall on lateral aspects of the uterus. The device 32 also advantageously provides for the intrauterine portion 40 expanding longitudinally outward, that is, axially distal. In the first variant shown in FIGS. 5A and 5B, the device includes a distal end portion 86, and the bladder 44 is not coupled to the distal end portion 86 or any other structure other than the base 56 of the bladder 44 being coupled to the conduit 36. As the first volume 52 receives the inflation fluid under pressure, the bladder 44 expands omnidirectionally; i.e., radially and axially. It is contemplated that a thickness of the bladder 44, and in particular the thickness of the tip portion 98 of the bladder 44 may be relatively greater to impart a desired expansion profile. The desired expansion profile may be more rounded or more flattened relative to the generally bulbous shape of the bladder 44.

In the second variant shown in FIGS. 6A and 6B, a distal cap 84 is movably disposed over the distal end portion 86 of the conduit 36. The distal end portion 86 of the conduit 36 may include a base 88 fixedly coupled to seal respective distal ends of the lumens or pathways of the conduit 36. The distal end portion 86 is tapered inwardly from the base 88. The distal cap 84 defines a cavity 90 sized to receive the distal end portion 86. More particularly, the distal cap 84 includes a trunk 92, and a cap head 94 extending distally from the trunk 92. The trunk 92 defines the cavity 90, and the cap head 94 may include a blunt tip 96. The bladder 44 is coupled to the distal cap 84, and in particular a tip portion 98 of the bladder 44 may be coupled to the cap head 94 of the distal cap 84. The cap head 94 may have an outer diameter less than an outer diameter of the trunk 92 such that the tip portion 98 being coupled thereto results in a generally smooth profile. It is noted that the outer body 58 may not be directly coupled to the distal cap 84.

As the first volume 52 receives the inflation fluid under pressure, the bladder 44 may expand omnidirectionally. The base 56 of the bladder 44 is fixedly coupled to the conduit 36 itself, yet the tip portion 98 of the bladder is fixedly coupled to the distal cap 84 and not the conduit 36. The distal forces from the axial expansion of the bladder 44 causes the distal cap 84 to move distally along the distal end portion 86 of the conduit 36 in which the bladder 44 is permitted to expand axially. In other words, the intrauterine portion 40 may widened and lengthened within the uterus. FIG. 6B shows a proximal end of the distal cap 84 being spaced apart from the 88 of the distal end portion 86. An extent by which the intrauterine portion 40 lengthened may be at least 10%, 15%, 20%, or 30% or more. The maximum extent by which the bladder 44 may be expanded axially is less than an initial distance by which the distal end portion 86 is positioned within the cavity 90 such that the distal cap 84 may not decouple from the conduit 36. The intrauterine portion 40 may be lengthened such that the distal end 68 of the device 32 contacts of the fundus of the uterus. In combination with the bulbous widening of the device, the shape of the device 32 advantageously may be contoured to nearly an entirety of the uterus to promote homeostasis with the tamponade-based treatment. In addition to the first variant, it is noted that in implementations in which the bladder 44 is formed from non-resilient, non-expandable material, the distal cap 84 may be optional, as the extent of axial expansion is based on a predetermined shape of the bladder 44.

With continued reference to FIGS. 4A-6B and with further reference to FIG. 8, the device 32 includes a collar 100. The collar 100 provides several benefits to be described. The collar 100 is coupled to the conduit 36. In particular, the collar includes an inner hub 102, an outer hub 104, and a barrier 106 coupling the inner hub 102 and the outer hub 104. The inner hub 102 may be directly connected to the conduit 36, for example, with an adhesive or other suitable joining means. The barrier 106 may extend radially outward from the inner hub 102 to the outer hub 104. The inner hub 102 may be proximal to the barrier 106, and the outer hub 104 may define a cavity 108 distal to the barrier 106. The cavity 108 is in fluid communication with the second volume 60, and the suction port 54 may be axially positioned within the cavity 108.

A base 110 of the outer body 58 is coupled to the collar 100 through adhesive or other suitable joining means or manufacturing techniques. In particular, the outer hub 104 may include a flange 112, and the base 110 of the outer body 58 may be coupled to a portion of the outer hub 104 that is distal to the flange 112. Moreover, the base 110 of the outer body 58 is coupled to the collar 100 at a position that is proximal to where the bladder 44 is coupled to the conduit 36, thereby establishing the fluid communication between the second volume 60 and the cavity 108. In other words, the second volume 60 may be considered to include the cavity 108.

Owing to the relative dimensions (e.g., outer diameters) of the collar 100 and the conduit 36, the base 110 of the outer body 58 is spaced apart from the conduit 36. The radial spacing provides multiple advantages over known devices in which a balloon is directly coupled to a conduit. First, the relatively greater initial width of the outer body 58 in the insertion configuration results in a smoother profile in the expanded configuration, thereby better accommodating the contours of the uterus immediately internal to the cervix. Second, the collar 100 provides a rigid structure configured to be gripped by the user during insertion, positioning, and/or repositioning of the device 32. The collar 100 may be formed from biocompatible materials with suitable stiffness to allow the user to grasp the collar 100 to support and manipulate the device 32. The collar 100 may include gripping features (e.g., texturization).

Third, in combination with the aforementioned distal cap 84, the collar 100 being fixedly coupled to the conduit 36 facilitates a stack up of subcomponents for improved manipulation of the device 32 by the collar 100. More particularly, the conduit 36 is fixedly coupled to the inner hub 102 of collar 100, and a proximal portion 114 of the conduit 36 (e.g., the portion defining the lumens) may be somewhat flexible to permit some lateral flexion of the conduit 36. The extent of lateral flexion may be 5, 10 or 15 or more degrees from a longitudinal axis of the device 32. Yet, the distal end portion 86 of the conduit 36 and the distal cap 84 may be relatively inflexible such that, with the intrauterine portion 40 in the insertion configuration and the user perhaps only grasping the device 32 by the collar 100, the subcomponent stack up prevents lateral “buckling” of the intrauterine portion 40 against resistance from the female anatomy. In other words, the extent of lateral flexion of the intrauterine portion 40 may be substantially limited to that afforded by the flexibility of the proximal portion 114 of the conduit 36. Still further, the struts 70 may also provide additional columnar strength or lateral stability to the device 32 during insertion, positioning, and repositioning. Taken together, the subcomponent stack up facilitates intuitive, ergonomic, and confident positioning of the device 32 in situ. At the same time, the subcomponent stack up provides some slack or “give” to prevent damage to sensitive tissue of the female anatomy and accommodate uteri of differing shapes.

Indicia (not shown) may be disposed on or coupled to the collar 100 to provide visual guidance to the user during insertion of the device 32. The indicia may be a colored marker or a marker visible under sonographic or fluoroscopic guidance. Additionally or alternatively, the collar 100 itself may be formed from a colored material. The colored material may be on the flange 112. During insertion, once the user can no longer see any aspect of the colored material, it may be assumed that the collar 100 is positioned just internal to the cervix, and the device 32 is in the appropriate position for deployment. Additional indicia may be provided to correspond to an anterior, upward-facing aspect of the device 32 visible to the physician as the device 32 is being positioned within the uterus should it be desired to appreciate the rotational orientation of the device 32 within the uterus.

The illustrated implementation shows the collar 100 defining suction openings 116. The suction openings 116 may extend through the outer hub 104, and more particularly be positioned within the flange 112. As a result, opposing edges of the flange 112 provide locating features for locating the base 110 of the outer body 58 and a head 118 of the cervical portion 42 to be described. The suction openings 116 are in fluid communication with the cavity 108, and further in fluid communication with the second volume 60. From FIGS. 5 and 6, it is also appreciated that an axial position of the suction openings 116 of the collar 100 is near the suction port 54 of the conduit 36. Owing to the positioning and size of the suction openings 116, the suction drawn on the uterus through the collar 100 may be relatively greater than that drawn through the suction openings 66 of the outer body 58. As generally appreciated from FIG. 1, the collar 100 is configured to be positioned within the uterus just internal to the cervix, and it has been shown that this location within the uterus is often prone to excessing pooling of blood during postpartum hemorrhage. Therefore, the device 32 advantageously addresses such concerns by providing less restricted suction at the most appropriate location. The perspective view of FIG. 8 shows the collar 100 as including four suction openings 116 equiangularly spaced radially about the flange 112. It is understood that more or less than four suction openings may be provided, and the suction openings 116 may be positioned in any regular or irregular manner about the collar 100.

Turning to the cervical portion 42, the cervical portion 42 is configured to be deployed to an expanded configuration to engage the cervix, and preferably seal the cervix. Engaging the cervical portion 42 facilitates accurately locating and fixing the position of the intrauterine portion 40 within the uterus. Doing so requires less observation and adjustment from attending medical personnel over known devices that must be manually supported. Further, the sealing of the cervix preserves optimal vacuum for the vacuum-based treatment. Referring again to FIGS. 5A-6B, the cervical portion 42 may include a seal body 120. The seal body 120 may include the head 118, and a base 122. The seal body 120 may be integrally formed through suitable biocompatible materials that are resiliently flexible. In such an implementation, the head 118 and the base 122 may demarcate portions of the seal body 120 that are coupled to other structures of the device 32.

FIGS. 5A and 5B show the cervical seal 42 and the collar 100 as an integrally-formed subcomponent, for which the collar 100 may be considered a distal collar portion of the cervical seal 42. This may be facilitated with suitable manufacturing techniques to impart the desired rigidity to the distal collar portion and the desired resiliency to the seal body 120. In the second variant shown in FIGS. 6A and 6B, the head 118 of the seal body 120 may be coupled to the collar 100, and the base 122 may be coupled to the conduit 36. In particular, the head 118 is coupled to the outer hub 104 of the collar 100 at a position that is proximal to the flange 112. The head 118 is fixedly coupled to the outer hub 104 with an adhesive or other suitable joining process or manufacturing technique. Likewise, the base 122 may be fixedly coupled to the proximal portion 114 of the conduit 36 with an adhesive or other suitable joining process or manufacturing technique.

In optional implementations, the cervical portion 42 may be configured to expand in an axially nonuniform manner. As best shown in FIG. 6, the seal body 120 may include a point of radialmost expansion 124 that, for convention, demarcates a proximal portion 126 and a distal portion 128 of the seal body 120. As used herein, axially nonuniform means that a contour or profile of the seal body 120 is different between the proximal portion 126 and the distal portion 128. The elevation sectional view of FIGS. 5B and 6B shows the proximal portion 126 as flaring proximally, whereas the radial contour of the distal portion 128 is relatively less pronounced. The axially nonuniform profile is imparted by the seal body 120 being of variable thickness along its length that is defined between the head 118 and the base 122. In particular, the proximal portion 126 of the seal body 120 has a first thickness (t₁), and the distal portion 128 has a second thickness (t₂) that is less than the first thickness. The axial location of the first thickness may be designed based on a desired axially nonuniform profile and/or the materials forming the seal body 120 (e.g., modulus of elasticity). The thinner aspects of the seal body 120 may be more likely to stretch than the thicker aspects of the seal body 120, and the thicker aspects may instead flex to impart the axially nonuniform profile. Among other advantages, the axially nonuniform profile provides improved engagement with and sealing of the cervix, particularly those that are postpartum in which the cervix has dilated and thinned.

The cervical portion 42 is configured to receive fluid to be deployed to the expanded configuration. The seal body 120 may define a third volume 130 in fluid communication with a second inflation pathway 50 defined by the conduit 36 (see also FIGS. 4A and 4B). The conduit 36 further defines a second inflation port 132 providing fluid communication between the second inflation pathway 50 and the third volume 130. The barrier 106 of the collar 100 may maintain fluid separation between the second volume 60 and the third volume 130. In other words, the second inflation port 132 may be proximal to the suction port 54, and it is also appreciated that the second inflation port 132 is proximal to the first inflation port 82. Therefore, according to one implementation of the device 32, the cervical portion 42 is configured to be deployed to the expanded configuration by directing or pumping fluid from the second fluid source through the second inflation pathway 50 and into the third volume 130. The extent to which the seal body 120 may be expanded is settable or selectable by the user and based on the clinical indications, and/or controlled through other means disclosed herein. As used herein, the expanded configuration means any widening or lengthening of the shape of the seal body 120 beyond its natural state, and therefore includes partial expansion as well as a maximum expansion permitted by the properties of the seal body 120.

The cervical portion 42 may be wider than the intrauterine portion 40 in the insertion configuration; however, the alternative arrangement is contemplated. In one example, a ratio of a maximum outer diameter of the intrauterine portion 40 to a maximum outer diameter of the cervical portion 42 is within a range of 0.7:1 and 1:0.9 in the insertion configuration. The intrauterine portion 40 being smaller may be particularly well suited in instances where the cervix is insufficiently dilated. The intrauterine portion 40 may be configured to have a maximum expansion greater than that of the cervical portion 42. For example, a ratio of a maximum outer diameter of the intrauterine portion 40 to a maximum outer diameter of the cervical portion 42 is within a range of 1.2:1 and 3.0:1 in the expanded configuration. The ratio may be based on the respective sizes, and/or the materials from which the intrauterine portion 40 and the cervical portion 42 are formed. For example, the intrauterine portion 40 may be formed from a first elastomer having a first modulus of elasticity, and the cervical portion 42 may be formed from a second elastomer having a different, second modulus of elasticity. Again, based on the intrauterine portion 40 and the cervical portion 42 being independently deployable to a partial or full extent, the device 32 of the present disclosure accommodates uteri of a range of shapes and sizes, and a range of stages of postpartum hemorrhage.

Referring now to FIGS. 9-12, another implementation of the device 32 is shown with like numerals indicating like components. In the illustrated implementation, the outer body 58 of the intrauterine portion 40 and the seal body 120 of the cervical portion 42 are a single-piece component of the device 32. In other words, the single-piece component of the device 32 may be manufactured to be monolithic or unitary in construction with the device 32 being shaped to a contoured profile complementary to the female anatomy. In such an arrangement, a medial taper (akin to the collar 100) may demarcate the intrauterine portion 40 and the cervical portion 42. The intrauterine portion 40 and/or the cervical portion 42 may be formed from multiple materials based on desired expansion and other characteristics of the device 32. For example, the intrauterine portion 40 may be formed from a first elastomer having a first modulus of elasticity, and the cervical portion 42 may be formed from a second elastomer having a different, second modulus of elasticity.

The intrauterine portion 40 and the cervical portion 42 are at least partially formed from flexible material, and more so from resiliently flexible material. The resilient material and/or shape of the device 32 may be specifically designed to provide sufficient columnar strength to facilitate insertion and positioning of the device 32 as desired. The device 32 may be sufficiently rigid to self-support its weight when grasped proximally, for example, manipulated by or near a proximal end of the cervical portion 42 or at the medial taper. The device 32 is also sufficiently flexible to be collapsible for insertion and then expandable once within the female anatomy. In other words, the present implementation of the device 32 may not be “floppy” akin to a conventional balloon-type device. The material characteristics and the contoured profile of the device 32 are configured to permit for better manipulation of the device 32 in which a distally-directed axial force may be applied to urge the intrauterine portion 40 through the cervix against resistance from the cervical os. As a result, the columnar strength advantageously provides for improved insertion through a caesarian opening, or through the cervix that may be smaller in size than an outer diameter of the device 32. It is contemplated, however, that the device 32 may include stiffening members (akin to the struts 70 or the spacing features 71) overmolded within or coupled to the outer body 58 of the intrauterine portion 40 to provide further columnar strength as desired.

Suitable materials for the intrauterine portion 40 and/or the cervical portion 42 may include polymers such as elastomers, nylons, resins, thermoplastics, thermosets, or the like, and exemplary manufacturing techniques include injection molding, blow molding, three-dimensional printing, among others. The materials may have an elastic moduli with the range of 0.01 to 2.0 Gigapascals (GPa), and more particularly within the range of 0.1 to 1.0 GPa. Suitable wall thicknesses may be within the range of 0.1 to 1.0 centimeters (cm), and more particularly within the range of 0.4 to 0.7 cm. The wall thickness may be based on the material characteristics so as to achieve the desired columnar strength and/or the contoured profile in the expanded configuration. For example, thicker portions of the device 32 may have a lower elastic modulus to permit greater radial expansion during deployment without compromising the columnar strength. The greater radial expansion of the intrauterine portion 40 is of particular interest to be contoured to wider, upper aspects of the uterus, yet be sufficiently flexible to conform to the uterus from resistance by the uterine wall.

The expansion profile of the device 32 is described with continued reference to FIGS. 9-12. The intrauterine portion 40 includes a head 134, a neck 136, and a crown 138. The neck 136 and the crown 138 extend from the head 134 in opposite directions. More particularly, the neck 136 extends proximally from the head 134, and the crown 138 extends distally from the head 134. As illustrated, the neck 136 may taper radially inwardly from the head 134, and the crown 138 may also taper radially inwardly to define the distal end 68 of the device 32. The distal end 68 may be blunt so as to avoid puncture other injury to the anatomy, while being contoured to distribute forces about the crown 138 during insertion. In such an arrangement, the intrauterine portion 40 may be considered generally bulbous in shape. With the intrauterine portion 40 in the insertion configured or original state, the head 134 may have an outer diameter that is least slightly greater than each of the neck 136 and the crown 138. The relative expansion of the head 134, the neck 136, and the crown 138 may be different such that, for example, the head 134 expands by a greater extent than the neck 136 and/or the crown 138. It is further contemplated that a kit may be provided (e.g., within a blister pack) in which there are several versions of the device 32 each having unique shape and/or expansion characteristics. A user may then select the optimal version most likely to be contoured to the shape of the uterus in either the default state and/or the expanded configuration.

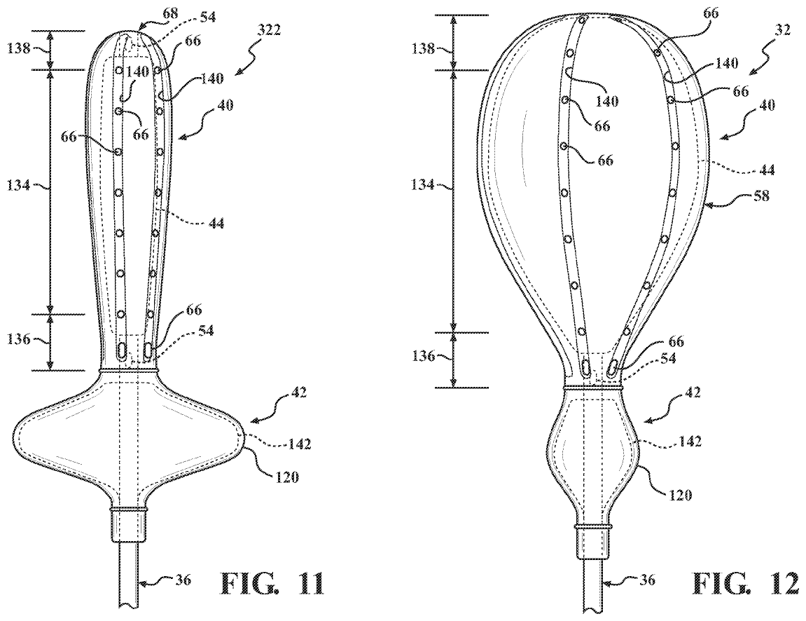

The outer body 58 of the intrauterine portion 40 defines the suction openings 66. The suction openings 66 may be arranged into rows spaced apart from one another and oriented radially about the intrauterine portion 40. One or more of the head 134, the neck 136, the crown 138 may not include the suction openings 66. The suction openings 66 are in a fluid communication with the second volume 60 defined between the inner surface 64 of the outer body 58, and the outer surface 62 of the bladder 44. The suction openings 66 are further in fluid communication with the conduit 36 at least partially extending through the intrauterine portion 40 and defining at least one suction port 54 in fluid communication with the suction openings 66. FIGS. 9 and 11 show the conduit 36 defining a proximal suction port and a distal suction port. The arrangement results in bodily fluids being able to pass through the suction openings 66, the second volume 60, and the suction ports 54 to drain or be drawn through the conduit 36. In other words, the intrauterine portion 40 may be positioned within the uterus with no vacuum to permit ingress of the bodily fluids through the device 32 (e.g., secondary to contractions of the uterus), or may be positioned with the uterus with vacuum applied to draw the bodily fluids through the device 32. In such an implementation, a drainage bag (not shown) may be coupled to the conduit 36, or to the control hub 34 to capture the patient fluids. The device 32 may be operated with or without suction regardless of whether the intrauterine portion 40 has been deployed to the expanded configuration. In other words, the intrauterine portion 40 may be operated with or without suction in the original state illustrated in FIGS. 9 and 11, and with or without suction in the expanded configuration illustrated in FIGS. 10 and 12.

The outer body 58 may further define at least one recess 140 with the suction openings 66 being disposed and in fluid communication with the recess 140. Disposing the suction openings 66 within the recess(es) 140 prevents or limits vacuum-based occlusion of the suction openings 66, thereby maintaining a desired vacuum strength and vacuum profile within the uterus. There may be one or more suction openings 66 within a respective one or more recesses 140. The subsequent disclosure is described with plural suction openings 66 within each of plural recesses 140. In the illustrated implementation, the recesses 140 are oriented longitudinally or substantially parallel to the longitudinal axis of the device 32. Distal ends of the recesses 140 may terminate proximal to the crown 138, or proximal to the distal end 68 of the device 32. Further, optionally, the recesses 140 may be radially arranged about the intrauterine portion 40, and the recesses 140 may be equiangularly arranged about the intrauterine portion 40. Still further, optionally, the suction openings 66 may be equally spaced within the recesses 140. The aforementioned arrangements may be particularly well suited when indicated to provide a uniform vacuum profile about the intrauterine portion 40.

Variations in positioning and geometries of the suction openings 66 and/or the recesses 140 are contemplated, namely those disclosed in connection with the previous implementation of the device 32. Further examples include a proximal one of the suction openings 66 being elliptical and larger than other suction openings 66. Any one or more of suction openings 66 may be circular, elliptical, oblong, and span any length within of the recess 140. The sizes, shapes, and/or positions of the suction openings 66 within the recess 140 may be modified to provide the desired vacuum profile about and about the device 32. There may be a greater or lesser concentration of the suction openings 66 disposed on the head 134, the neck 136, or the crown 138. A higher concentration of the suction openings 66 within the head 134 may be provided to correspond to the anatomical junction between the uterine arteries and the uterus. Regarding the geometries of the recesses 140, the recesses 140 may be elongate and generally linear as shown. A variation includes recesses extending circumferentially about the head 134, for example, in addition to the longitudinal recesses to provide a grid. Further variations include the recesses 140 being zig-zagged, helical, or the like.

The recesses 140 may be equiangularly spaced about the intrauterine portion 40. An alternative implementation includes the recesses 140 asymmetrically arranged based on anatomical areas of higher anticipated bleeding. For example, with the patient lying supine, blood and other bodily fluids may be more likely to collect posteriorly within the uterus due to the influence of gravity. Therefore, it is contemplated that a higher concentration of the suction openings 66 may be provided on a posterior portion (not identified) of the intrauterine portion 40 when oriented in a predetermined insertion orientation. Indicia may be provided on a proximal component of the device that corresponds to an anterior, upward-facing aspect of the device 32 visible to the physician as the device 32 is being positioned within the uterus. Orienting the indicia upwardly locates the greater concentration of the suction openings 66 posteriorly. Other arrangements of the suction openings 66 to promote the desired vacuum profile are within the scope of the present disclosure.

In the present implementation, the bladder 44 may expand uniformly with the outer body 58 constraining expansion to provide the contoured profile. Alternatively, the bladder 44 may be designed to provide the contoured profile with the outer body 58 conforming a shape of the bladder 44 with little resistance. In one variant, the bladder 44 may include a plurality of bladders each configured to be independently deployed to provide the contoured profile to the outer body 58. For example, a separate bladder may be disposed within each of the head 134, the neck 136, and the crown 138 to provide more pronounced or varied contouring to the outer body 58. The intrauterine portion 40 may be configured may be expand axially and radially. In other words, the material and dimensions of the intrauterine portion 40 may be designed such that, with the bladder 44 being filled with the fluid, the device 32 is lengthened and widened within the uterus. An extent by which the device 32 lengthens may be at least 5%, 10%, 15%, or 20% or more. The intrauterine portion 40 may be lengthened such that the distal end 68 contacts of the fundus of the uterus.

One or both of the bladder 44 and the outer body 58 may include the spacing features 71 to maintain a slight gap therebetween despite the bladder 44 urging the outer body 58 to expand. The spacing features 71 may be nubs, projections, ridges, or contours defined in the bladder 44 and/or the outer body 58. The suction path is further defined by the suction ports 54, and in particular the proximal suction port 54. The proximal suction port 54 positioned within the outer body 58 and proximal to a proximal end of the bladder 44. Therefore, the proximal suction port 54 remains patent with expansion of the bladder 44 within the outer body 58. The vacuum source draws the blood from within the uterus, through the suction openings 66, along the second volume 60 between the bladder 44 and the outer body 58, and through the proximal suction port 54. It is further noted that the proximal suction port is optimally positioned within the lower uterus to better evacuate the blood that may accumulate in the anatomical area.

In the present implementation, the cervical portion 42 may be deployed by filling another bladder 142 with fluid, such as saline, to force expansion of the seal body 120 to the expanded configuration such that the intrauterine portion 40 and the cervical portion 42 are independently deployable. FIG. 9 illustrates the intrauterine portion 40 as undeployed and the cervical portion 42 as undeployed, for example, during initial positioning of the device 32. FIG. 10 illustrates the intrauterine portion 40 as deployed and the cervical portion 42 as deployed configuration, for example, during the tamponade-based (and, optionally, the suction-based) treatment. FIG. 11 illustrates the intrauterine portion 40 as undeployed and the cervical portion 42 as deployed. FIG. 12 illustrates the intrauterine portion 40 as deployed and the cervical portion 42 as undeployed. The intrauterine portion 40 may be smaller or larger than the cervical portion 42, and further may be expanded by a lesser or greater extent than the cervical portion 42. In one example, a ratio of a maximum outer diameter of the intrauterine portion 40 to a maximum outer diameter of the cervical portion 42 is within a range of 0.8:1 and 1:1.2 in the original state. In other words, when undeployed, the intrauterine portion 40 may be slightly narrower to slight wider than the cervical portion 42. In another example, the ratio of a maximum outer diameter of the intrauterine portion to a maximum outer diameter of the cervical portion may be within a range of 1:2.0 and 1:4.0 in the original state. It may be indicated for the intrauterine portion 40 to be sufficiently collapsible to be inserted through the opening despite the relatively larger outer diameter. Conversely, the intrauterine portion 40 may be narrowed by removing initial air within the bladder 44, after which the resilient material forming the intrauterine portion 40 returns to the original state. Likewise, the cervical portion 42 may be narrowed by removing initial air from the bladder 142.

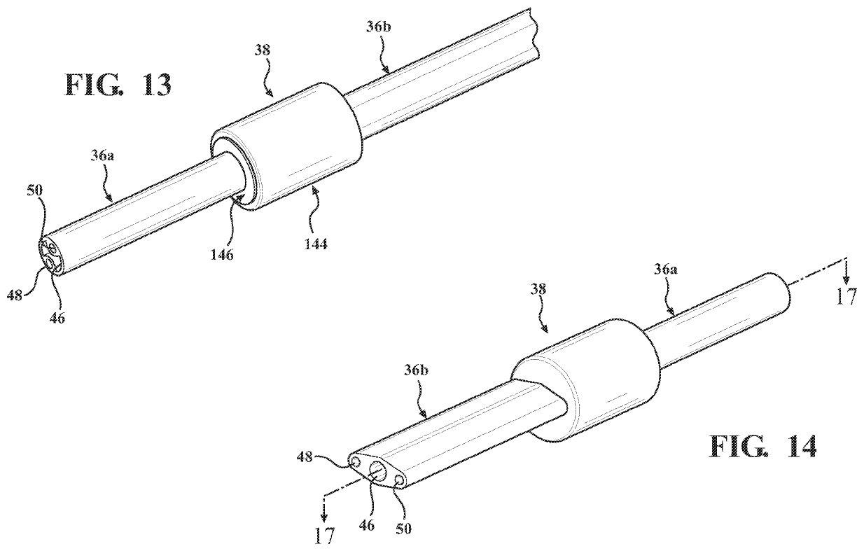

As mentioned, the suction pathway 46 is configured to be arranged in fluid communication with the vacuum source, the first inflation pathway 48 is configured to be arranged in fluid communication with the first fluid source, and the second inflation pathway 50 is configured to be arranged in fluid communication with the second fluid source. One manner by which this may be accomplished is through the connector 38 and the control hub 34 each to be discussed in turn. Referring to FIGS. 13 and 14, the connector 38 facilitates removable coupling of a first section 36a and a second section 36b of the conduit 36. The first section 36a may extend to and include the proximal portion 114 of the conduit 36 previously discussed (see FIGS. 5 and 6). The connector 38 includes a female component 144 fixedly or removably coupled to the second section 36b, and a male component 146 fixedly or removably coupled to the first section 36a.

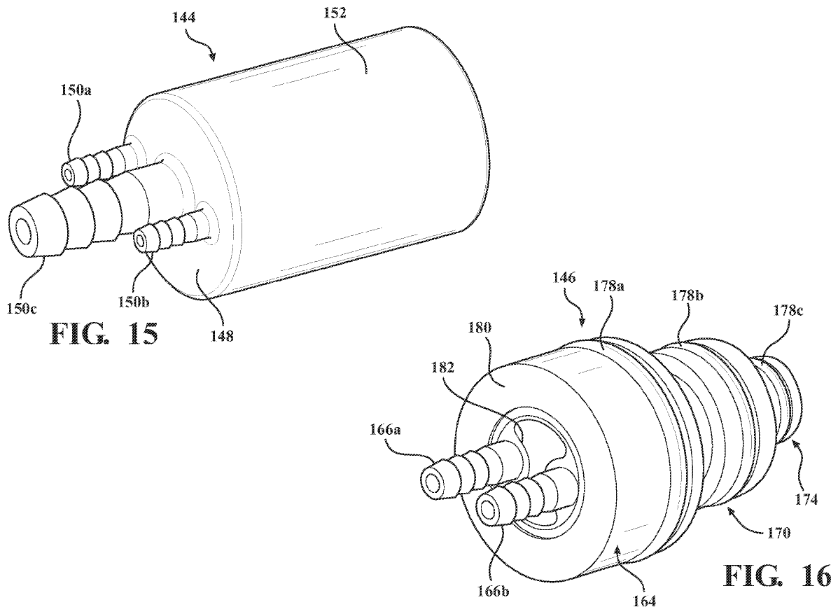

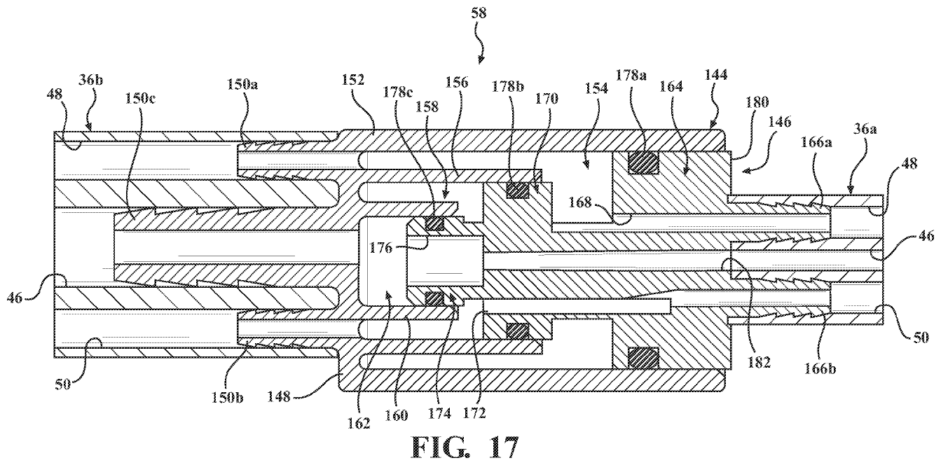

FIGS. 15 and 17 show the female component 144 including an endwall 148, and at least one fitting 150 extending from the endwall 148 to define a first end of the female component 144. An outer sidewall 152 extends from the endwall 148 to define a second end of the female component 144. The outer sidewall 152 defines a first cavity 154. A first inner wall 156 is coaxially disposed within the outer sidewall 152. The outer sidewall 152 extends from the endwall 148 by a distance less than a distance by which the outer sidewall 152 extends from the endwall. The first inner wall 156 has a first inner diameter and defines a second cavity 158 within the first cavity. The female component 144 includes a second inner wall 160 coaxially arranged within the first inner wall 156 and extending from the endwall 148. The second inner wall 160 extends from the endwall 148 by a distance less than the distance by which the first inner wall 156 extends from the endwall 148. The second inner wall has a second inner diameter and defines a third cavity 162 within the second cavity 158.

The male component 146 includes a first section 164 having a first outer diameter sized to be received within the first cavity of the female component. The first section 164 includes at least one fitting 166 and defines a first offset bore 168 configured to be arranged in fluid communication with the first cavity 154 of the female component 144. A second section 170 is coaxially arranged relative to the first section 164 and has a second outer diameter sized to be received within the second cavity 158 of the female component 144. The second section 170 defines a second offset bore 172 configured to be arranged in fluid communication with the second cavity 158 of the female component 144. A third section 174 is coaxially arranged relative to the second section 170 and has a third outer diameter sized to be received within the third cavity 162 of the female component 144. The second section 170 defines a central bore 176 arranged in fluid communication with the third cavity 162 of the female component 144. A first seal 178a coupled to the first section 164, a second seal 178b coupled to the second section 170, and a third seal 178 coupled to the third section 174.

The fitting 166 of the male component 146 may be a first fitting 166a in fluid communication with the first offset bore 168, and a second fitting 166b in fluid communication with the second offset bore 172. A face 180 of the male component 146 defines a noncircular opening 182 between the first fitting 166a and the second fitting 166b with the noncircular opening 182 in fluid communication with the central bore 176. On the female component 144, the fitting 150 may be a first lateral fitting 150a in fluid communication with the first cavity 154, a second lateral fitting 150b in fluid communication with the second cavity 158, a third central fitting 150c in fluid communication with the third cavity 162. The third central fitting 150c may be larger than the first and second lateral fittings 150a, 150b.

The conduit 36 defines the suction pathway 46, the first inflation pathway 48, and the second inflation pathway 50, and thus each of the first and second sections 36a, 36b define portions of the pathways 46, 48, 50 when removably coupled together via the connector 38. As best shown in FIGS. 4A, 4B, 13 and 14, the lumens of the conduit 36 are of different sizes. In particular, the effective cross-sectional area of the suction pathway 46 is greater than an effective cross-sectional area of each of the first and second inflation pathways 48, 50. In other words, the conduit 36 may have a constant outer diameter, and a relatively greater cross-sectional area of the conduit 36 is dedicated to the suction pathway 46. Such an arrangement advantageously recognizes the often-greater demand for suction. To facilitate the arrangement yet minimize the outer diameter of the conduit 36, the suction pathway 46 may be noncircular with the illustrated implementation being one non-limiting example. The relative cross-sectional areas may be such that the effective cross-sectional area of the suction pathway 46 is within the range of 0.50 to 0.65 square inches. With the second section 36b of the conduit 36 extending between the connector 38 and the control hub 34, and thereby positioned more remote from the patient, minimizing the outer diameter may be of less concern. In the illustrated implementation, the second section 36b may be wide and thin to be ribbon-like in shape with the first and second inflation pathways 48, 50 positioned opposite the suction pathway 46 being of larger diameter.

The second section 36b of the conduit 36 is removably coupled to the female component 144 of the connector 38. More particularly, the first inflation pathway 48 is coupled to the first fitting 150a, the second inflation pathway 50 is coupled to the second lateral fitting 150b, and the suction pathway 46 is coupled to the third central fitting 150c. Likewise, the first section 36a of the conduit 36 is removably coupled to the male component 146 of the connector 38. The first inflation pathway 48 is coupled to the first fitting 166a, and the second inflation pathway 50 is coupled to the second fitting 166b. A corresponding fitting (not identified) of the first section 36a is arranged in fluid communication with the noncircular opening 182.

In an alternative implementation, the cavities defined by the connector 38 may not be coaxial, but laterally offset from one another. The first and second sections 36a, 36b of the conduit 36 are correspondingly modified to match the lateral offset of each of the suction pathway 46, the first inflation pathway 48, and the second inflation pathway 50.

When desired or otherwise indicated, the female component 144 and the male component 146 are coupled to one another in the aforementioned manner and as illustrated in FIG. 17. The arrangement establishes fluid communication of the suction pathway 46 via the central bore 176 and the third cavity 162, of the first inflation pathway 48 via the first offset bore 168 and the first cavity 154, and of the second inflation pathway 50 via the second offset bore 172 and the second cavity 158. The connector 38 facilitates a quick, intuitive, and compact removable coupling between the sections 36a, 36b of the conduit 36 having at least three pathways. Such advantageous may be particularly useful for certain procedures and workflows. For example, the device 32 is also configured to be used with Caesarian sections in which the device 32 is directed through the Caesarian opening as opposed to the cervix. The outer diameter of the female component 144 of the connector 38 is sufficiently small to be passed through the cervix even if not more than minimally dilated. The connector 38 is further passed through and external to the vagina, after which it may be removably coupled to the male component 146 of the connector 38. The step of coupling may occur before or after the device 32 is positioned as desired within the female anatomy. For another example involving vaginal births and Caesarian sections, the connector 38 permits a primary user to be performing initial placement of the device 32 within the female anatomy, and a secondary user to be arranging the conduit 36, the connector 38, and the control hub 34, thereby improving efficiency of the surgical workflow.

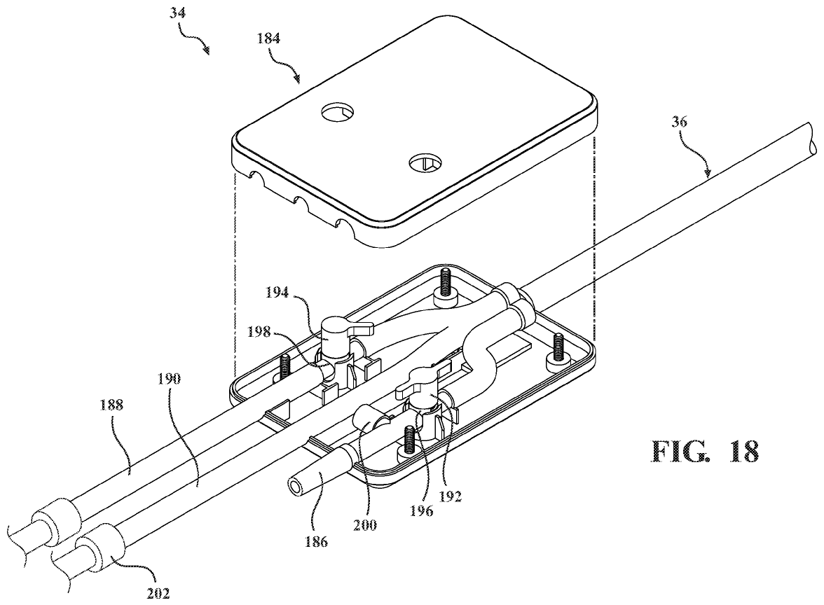

Referring now to FIG. 18, the control hub 34 includes a module housing 184 coupled to a vacuum line 186, a first supply line 188, and a second supply line 190. In a broadest sense, the control hub 34 is subcomponent of the system 30 configured to control the various functionality of the device 32 described throughout the present disclosure. More particularly, the control hub 34 functions as an intuitive hub for selectively and individually controlling deployment the intrauterine portion 40, deploying the cervical portion 42, and vacuum being drawn through the device 32. The control hub further provides for improved line management over known devices in which tubes extend from a device in a disorganized and unwieldly manner. The control hub 34 includes a first user input 192, and may further include and a second user input 194 that is optional. The first user input 192 is configured to receive an input to actuate a vacuum valve 196 between an open position in which the vacuum on the suction openings 66 from the vacuum source is permitted, and a closed position in which the vacuum on the suction openings 66 from the vacuum source is prevented. In other words, the first user input 192 actuates on and off the vacuum through the device 32, and further may be actuated to maintain the vacuum within the device 32 during patient transport. The second user input 194 may be configured to receive another input to actuate a first inflation valve 198 to permit or prevent fluid passing through the first supply line 188 to within the bladder 44 of the intrauterine portion 40.

As generally appreciated from FIG. 18, the vacuum line 186, the first supply line 188, and the second supply line 190 are configured to be coupled to the conduit 36. The coupling may be integral such that the lines 186, 188, 190 are continuous with the conduit 36, or the module housing 184 may include a plug or port configured to be removably coupled with the conduit 36. The vacuum valve 196 is configured to be arranged in fluid communication with the suction pathway 46 of the conduit 36, and the first inflation valve 198 configured to be arranged in fluid communication with the first inflation pathway 48 of the conduit 36. The first user input 192 may be a first switch operably coupled to the vacuum valve 196 and configured to receive a first input to actuate the vacuum valve 196 to permit or prevent suction from a vacuum source to be drawn on the second volume 60. The second user input 194 may be a second switch operably coupled to the first inflation valve 198 and configured to receive a second input to actuate the first inflation valve 198 to a closed position to maintain inflation of the bladder 44 with fluid in the first volume 52 in a manner to be further described. Additionally or alternatively, the user inputs may be knobs as shown, or electronically-actuated buttons or switches.

The control hub 34 may include a second inflation valve 202 configured to be arranged in fluid communication with the second inflation pathway 50 of the conduit 36. The second inflation valve 202 is operable to permit or prevent fluid from the second fluid source to be directed to or removed from the second volume 60, thereby deploying and undeploying the cervical portion 42, respectively. The second inflation valve 202 may be a self-sealing fitting configured to be coupled with the second inflation source. For example, the second inflation valve 202 may be a self-sealing Luer fitting configured to be coupled with a fluid source such as a syringe of saline. In a variant in which the second user input 194 is not provided, the second inflation valve 198 may also be a self-sealing Luer fitting configured to be coupled with another syringe of saline. In still another variant, a toggle switch (not shown) may be provided in fluid communication with each of the first and second supply lines 188, 190. A single fluid source (e.g., syringe of saline) may be coupled to the control hub 34, and the toggle switch actuated in a manner to selectively provide the fluid to either the intrauterine portion 40 or the cervical portion 42. The control hub 34 may include an optional suction relief valve 200 configured to break the vacuum if the vacuum level exceeds a predetermined threshold.

The module housing 184 may include indicia on a user-facing face to provide information instructive as to which of the first and second user inputs 192, 194 correspond to which of the operations of the device 32. Exemplary indicia may be schematic representation of the female anatomy, and/or a schematic representation of the device 32. Additionally or alternatively, indicia may be provided on the first supply line 188 and/or the second supply line 190 to indicate to which portion of the device 32 the fluid is to be directed. For example, a “U” may be etched or printed on the first supply line 188 to indicate uterus, and a “C” may be etched or printed on the second supply line 190 to indicate cervix. The module housing 184 may be smaller in form factor so as to be affixed to a patient support apparatus or the leg of patient. A clip (not shown) may be coupled to the conduit 36 and configured to be coupled to a bedsheet, and/or a hook assembly (not shown) may be coupled to the conduit 36 and configured to be coupled to a rail or other structure of the patient support apparatus. The module housing 184 may be disposable such that sterilization is not indicated and the module housing 184 may be positioned within the sterile field if necessary.

Exemplary methods of treating the uterus, particularly bleeding of the uterus during postpartum hemorrhage, include the step of providing the system 30 described herein. The system 30 may be provided in a sterile blister pack. The device 32 and the control hub 34 are removed from the blister pack. The connector 38 may or may not be already coupled to the first section 36a of the conduit 36 that extends to the device 32.

The intrauterine portion 40 is in the insertion configuration, and the cervical portion 42 is in the insertion configuration. For a workflow involving transvaginal insertion, the primary user may grasp the device 32 by the collar 100, and direct the distal end 68 of the intrauterine portion 40 through the vagina, and further through the cervix. The subcomponent stack up previously described facilitates intuitive and ergonomic insertion. The primary user may observe the indicia on the collar 100 to position the device 32 appropriately relative to the cervix. In a workflow involving Caesarian sections, the primary user may direct the conduit 36 through the caesarian opening and outwardly through the cervix and the vagina. The cervical portion 42 is positioned appropriately relative to the cervix from within.

Either prior to, during, or after the step of the primary user positioning the device 32 within the female anatomy, the secondary user may couple the first section 36a of the conduit 36, if necessary. The second section 36b of the conduit 36 may be coupled to the control hub 34, if necessary. The female component 144 and the male component 146 of the connector 38 are coupled. Either prior to, during, or after the step of coupling the female component 144 and the male component 146 of the connector 38, the vacuum line 186 of the control hub 34 is coupled to the vacuum source. A fitting at the end of the vacuum line 186 may receive a suction tube from the vacuum source via friction or interference fit. The vacuum valve 196 may in the closed position. The first supply line 188 of the control hub 34 is coupled to the first fluid source. The first fluid source may be a syringe of saline. A Luer fitting at the end of the first supply line 188 may facilitate the coupling. The syringe remains filled with the saline with the first inflation valve 198 in the closed position. The second supply line 190 of the control hub 34 is coupled to the second fluid source. The second inflation valve 202 may be a self-sealing Luer fitting configured to be coupled with the second fluid source, namely a second syringe of saline.

After confirming proper positioning of the cervical portion 42, the second syringe of saline is actuated to direct the fluid through the second supply line 190, the second inflation pathway 50 of the conduit 36, and into the third volume 130 defined by the seal body 120. Graduations on the second syringe may provide an indication of the volume of saline being directed, and a corresponding amount of expansion may be understood based on known conversions. Additionally or alternatively, the user may feel resistance in the second syringe to indicate the cervix being engaged by the seal body 120. The cervical portion 42 is deployed to the expanded configuration against the cervix. The second syringe is decoupled from the self-sealing Luer fitting, after which the cervical portion 42 is maintained in the expanded configuration.

If desired to provide the tamponade-based treatment, the intrauterine portion 40 is deployed. The user actuates the second user input 194 to move the first inflation valve 198 the open position, and further actuates the first syringe of saline to direct the fluid through the first supply line 188, the first inflation pathway 48 of the conduit 36, and into the first volume 52 defined by the bladder 44. The intrauterine portion 40 is deployed to the expanded configuration. Graduations on the first syringe may provide an indication of the volume of saline being directed, and a corresponding amount of expansion may be understood based on known conversions. Additionally or alternatively, the user may feel resistance in the first syringe to indicate the uterine wall being engaged by the outer body 58. If necessary, with the first inflation valve 198 in the open position, the user may retract the first syringe to draw the saline from the first volume 52. Once satisfied with the extent of expansion, the user actuates the second user input 194 to move the first inflation valve 198 to the closed position, thereby maintaining the volume of fluid within the bladder 44 and the intrauterine portion 40 in the expanded configuration.

If desired to provide the vacuum-based treatment—with or without the intrauterine portion 40 deployed—the user may perform the step of operating the vacuum source. The vacuum source may be activated with the vacuum valve 196 in the closed position, at least initially. The user may actuate the first user input 192 to move the vacuum valve 196 at least partially towards the open position. The vacuum is drawn through the suction openings 66, the second volume 60, the suction pathway 46 of the conduit 36, and the vacuum line 186. Should there be any unexpected spikes in the level of vacuum, the suction relief valve 200 may open. Otherwise, the vacuum level may be adjusted by further inputs to the first user input 192 and/or to the system controlling the vacuum source.

With the tamponade-based and vacuum-based treatments, the uterus may desirably begin to contract and shrink. The method may include deflating the bladder 44 to decrease the extent of expansion of the intrauterine portion 40 in a manner corresponding to the shrinking of the uterus. The user may provide another input to the second user input 194 to move the first inflation valve 198 to the open position, and further retract the first syringe of saline to draw the fluid out of the bladder 44. Additionally, the user may adjust the vacuum level as the bladder 44 as this is occurring. For example, with the intrauterine portion 40 fully deployed, a higher or lower vacuum level may be indicated based on the contractions being induced. As the bladder 44 is deflated, the vacuum level may be increased or decreased by a predetermined or selected amount. For another example, the vacuum level may be maintained while the bladder 44 is deflated by a fixed or varied amounts at fixed or varied intervals.

The device 32 may be manufactured according to exemplary methods. The collar 100 is arranged to be slidable over the conduit 36, and secured in position along the conduit 36. The bladder 44 is secured to the distal cap 84. The distal cap 84, with the bladder 44 coupled thereto, to be slidable over the distal end portion 86 of the conduit 36. The base 56 of the bladder 44 is secured to the conduit 36 to define the first volume 52. The outer body 58 is positioned over the bladder 44, and secured to the collar 100 to define a second volume 60 between the bladder 44 and the outer body 58.

In certain methods, the struts 70 are coupled to the inner surface 64 of the outer body 58. For example, the struts 70 may be positioned in a jig, and the outer body 58 overmolded on the struts 70. The seal body 120 of the cervical portion 42 may be secured to the collar 100 of the conduit 36 to define the third volume 130. The conduit 36 may be extruded to define lumens of different cross-sectional areas, for example, in which a cross-sectional area of a largest of the lumens is within the range of 0.50 to 0.65 square inches.

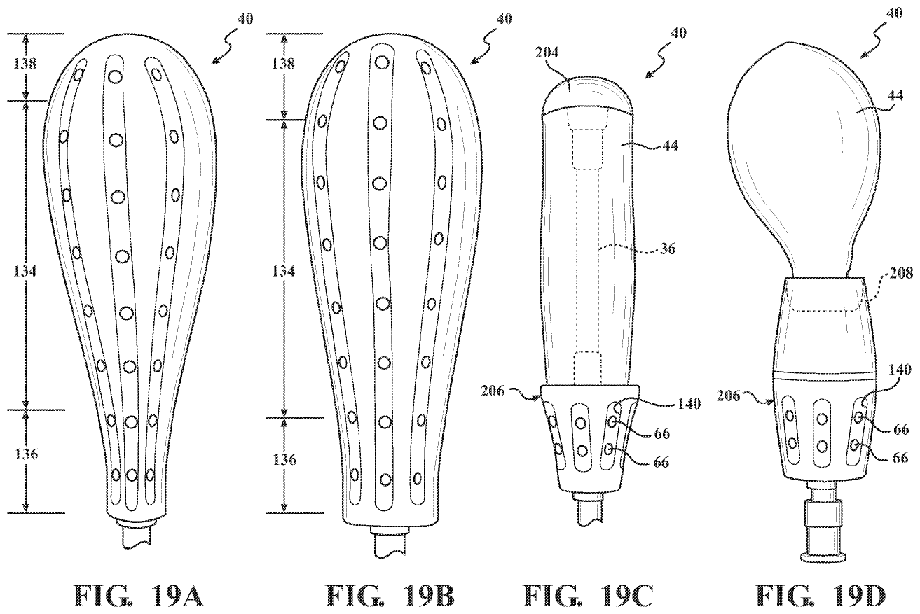

FIGS. 19A-19D illustrate contemplated alternative implementations of the intrauterine portion 40. FIG. 19A illustrates a dimensional alternative, relative to FIG. 9, in which the head 134 is wider. FIG. 19B illustrates another dimensional alternative, relative to FIG. 9, in which the head 134 and the neck 136 are wider, wherein the neck 136 is wider than that of FIG. 19A. The neck 136 being wider may promote improved engagement with the lower uterine walls, which are typically less elastic than the fundus of the uterus. FIG. 19C illustrates an alternative in which the bladder 44 is disposed between a cap 204 and a base 206. The suction openings 66 may be disposed on the base 88. FIG. 19D illustrates an alternative in which the bladder 44 expands radially and distally outward from the base 206. The bladder 44 may be initially seated within a cavity 208 so as to be exposed above the base 206 when deployed.

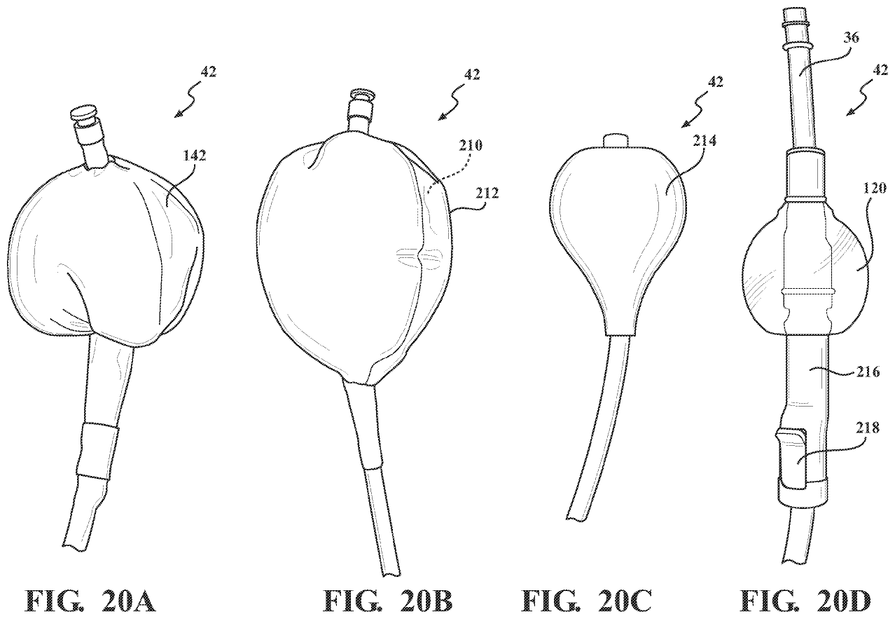

FIGS. 20A-20C illustrate contemplated alternative implementations of the cervical portion 42. FIG. 20A illustrates the bladder 142 configured to be inflated with a fluid. FIG. 20B is configured to self-inflate by including a compressible body 210 within a casing 212. The compressible body 210 may be formed from foam or another suitable material. The compressible body 210 is configured to be squeezed, and upon release of the input, permit ingress of air to expand. A valve, such as a stopcock, may be provided to prevent further expansion of the compressible body 210 in the desired dimension. FIG. 20C illustrates a squeeze bulb formed from resiliently flexible material. The squeeze bulb is configured to be squeezed, and upon release of the input, expand towards its original shape. A valve may prevent further expansion of the squeeze bulb in the desired dimension. FIG. 20D illustrates the seal body 120 coupled to a housing 216. An actuator 218 may be coupled to the housing 216, and configured to receive an input to move the intrauterine portion 40 relative to the seal body 120 by sliding the conduit 36 (to which the intrauterine portion 40 is coupled) relative to the housing 216.

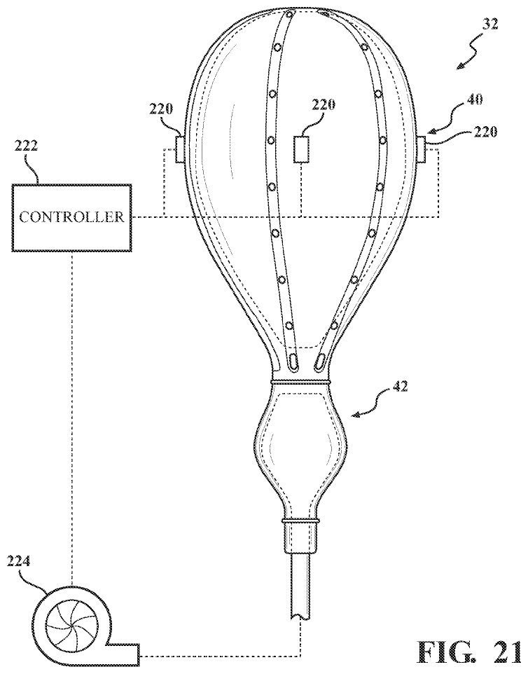

Referring now to FIG. 21, the device 32 may include at least one sensor 220 arranged in wired or wireless communication with a processor or controller 222. The sensor 220 may be disposed on the intrauterine portion 40, or the cervical portion 42, or both. The sensor 220 is configured to generate and transmit a signal to the controller 222 to determine a force being applied to the uterine wall by the device 32. This obviates the need to rely on vacuum level readings for potentially less accurate determinations of the extent of engagement between the device 32 and the uterus.

The sensor 220 may be plurality of sensors disposed about the outer body 58 of the intrauterine portion 40. The sensors 220 may be contact sensors, force sensors, displacement sensors, or the like. The contact sensors may be configured to transmit a contact signal to a controller 222 or an output device with the signal indicative that the head 134 is contacting a uterine wall of the uterus. In instances of manual deployment of the intrauterine portion 40, the contact signal may be displayed on the output device to inform the user that the outer surface is engaging the uterine wall. In an alternative arrangement, the system 30 may include a pump 214 in communication with the controller 222 with the controller 222 configured to operate the pump 214 to deploy the intrauterine portion 40 with until the contact signal is received. Additionally or alternatively, the force sensors may be configured to transmit a force signal to the controller 222 indicative of a magnitude of force on from the device 32 on the uterine wall, and therefore the force on the uterine wall from the device 32. An exemplary force sensor includes a strain gauge, a load cell, among others. Additionally or alternatively, the displacement sensors may be configured to transmit to a displacement signal to the controller 222 indicative of an extent of expansion of the intrauterine portion 40. As the uterus is contracting as desired, the extent of expansion of the intrauterine portion 40 may be outputted on the output device to indicate improvement of patient status. The sensors 220 and the controller 222 may facilitate operating the tamponade and the suction-based treatments in the independent or codependent manners previously described. It is further contemplated that the sensors 220 are optional, and the characteristics of the operation of the device 32 may be obtained through other means, such as electronic sensing and feedback of the medical waste collection system.

Additionally or alternatively, the user may selectively set one of the magnitude of expansion and the vacuum level with the other being automatically controlled by a control protocol stored on memory in communication with the controller. Still further, the user may select a preprogrammed control protocol stored on memory in communication with the controller, and the controller may control the magnitude of expansion and the vacuum level accordingly. An exemplary method includes adjusting the vacuum level as the bladder 44 is deflated (i.e., the magnitude of expansion of the intrauterine portion 40 is decreased). For example, with the intrauterine portion 40 fully deployed, a higher or lower vacuum level may be indicated based on the contractions being induced. As the bladder 44 is deflated and the intrauterine portion 40 “shrinks” with the contracting uterus, the vacuum level may be increased or decreased by a predetermined or selected amount. For another example, the vacuum level may be maintained while the bladder 44 is deflated by a fixed or varied amounts at fixed or varied intervals.

Certain aspects of the present disclosure are described with reference to the following exemplary clauses:

Clause 1—A device for treating a uterus, the device comprising a conduit defining a suction pathway and an inflation pathway; a bladder coupled to the conduit and defining a first volume in fluid communication with the inflation pathway, wherein the conduit defines a suction port external to the bladder with the suction port in fluid communication with the suction pathway.

Clause 2—The device of clause 1, wherein an outer surface of the bladder further comprises spacing features configured to maintain separation between the bladder and the uterus.

Clause 3—The device of clause 1 or 2, wherein the suction port is positioned proximal to a base of the bladder or distal a tip portion of the bladder.

Clause 4—The device of clause 3, wherein the conduit further defines an inflation port positioned distal to the suction port and distal to the base of the bladder.

Clause 5—A device for treating a uterus, the device comprising a conduit defining a suction pathway; an outer body defining a volume in fluid communication with the suction pathway, and comprising an outer surface defining suction openings in fluid communication with the volume, wherein the conduit defines a suction port external to the bladder with the suction port in fluid communication with the suction pathway.

Clause 6—A device for treating a uterus, the device comprising: a conduit defining a suction pathway and an inflation pathway; a bladder defining a first volume in fluid communication with the inflation pathway; and an expandable outer body defining suction ports in fluid communication with the suction pathway, wherein a channel is defined between an inner surface of the outer body and an outer surface of the bladder with the channel providing fluid communication between the suction ports the suction pathway.

Clause 7—The device of clause 6, further comprising a strut coupled to at least one of the inner surface of the outer body or a spacing feature integrally formed with the inner surface of the outer body, wherein the strut or the spacing feature defines the channel.

Clause 8—The device of clause 7, wherein the strut or the spacing features comprises projections defining the channel therebetween, wherein the opposing projections is configured to maintain separation between the bladder and the outer body for bodily fluids to be drawn through the suction ports and into the suction pathway.

Clause 9—The device of clause 7 or 8, wherein the strut defines bores aligned with the suction ports of the outer body.

Clause 10—The device of clause 9, wherein the bores are positioned within the channel.

Clause 11—The device of any one of clauses 7-9, wherein the strut is oriented longitudinally along an inner surface of the outer body.

Clause 12—The device of any one of clauses 7-10, wherein a distal portion of each of the struts is preformed with a bend to flare radially inwardly.

Clause 13—The device of any one of clauses 7-12, wherein the outer body is overmolded on the strut; and, optionally, wherein the strut is otherwise not directly connected to another subcomponent of the device.

Clause 14—The device of any one of clauses 7-13, wherein the strut further comprises at least three struts equiangularly spaced radially about the conduit.

Clause 15—A device for treating a uterus, the device including a conduit defining an inflation pathway; a distal cap movably disposed over a distal portion of the conduit; and a bladder comprising a base coupled to the conduit, and a tip portion coupled to the distal cap, wherein the bladder defines a volume in fluid communication with the inflation pathway, wherein the distal cap is configured to move distally along the distal portion of the conduit in response to the bladder being expanded with fluid directed from the inflation pathway and into the volume.

Clause 16—The device of clause 15, wherein the conduit defines a suction pathway separate from the inflation pathway, wherein the device further comprises an outer body coupled to the conduit and at least partially disposed over the bladder, wherein the outer body defines suction ports, and wherein a second volume defined between the bladder and the outer body is in fluid communication with the suction ports and the suction pathway of the conduit.

Clause 17—The device of clause 15 or 16, wherein the distal cap comprises a trunk, and a cap head extending distally from the trunk, wherein the cap head has an outer diameter less than an outer diameter of the trunk, and wherein the tip portion of the bladder is coupled to the cap head.

Clause 18—The device of clause 17, wherein the trunk of the distal cap defines a cavity sized to receive the distal portion of the conduit.

Clause 19—The device of clause 17 or 18, wherein the cap head comprises a blunt distal end.

Clause 20—A device for treating a uterus, the device including a conduit defining a suction pathway and an inflation pathway; a bladder coupled to the conduit and defining a volume in fluid communication with the inflation pathway; an outer body defining suction ports in fluid communication with the suction pathway; and a collar coupled to the conduit, wherein a base of the outer body is coupled to the collar at a position proximal to where the bladder is coupled to the conduit.

Clause 21—The device of clause 20, wherein the base of the bladder is spaced apart from the conduit.

Clause 22—The device of clause 20 or 21, wherein the conduit comprises a distal end portion that is inflexible, wherein the conduit being fixedly coupled to the collar provides a subcomponent stack up to prevent buckling of the intrauterine portion during insertion of the device while being grasped by the collar.

Clause 23—The device of any one of clauses 20-22, further comprising an expandable cervical seal coupled to the collar and the conduit.

Clause 24—The device of any one of clauses 20-23, wherein the collar defines additional suction ports in fluid communication with the suction pathway.

Clause 25—The device of any one of clauses 23 or 24, wherein the collar comprises a flange that defines the additional suction ports, wherein the bladder and the cervical seal are coupled on opposing sides of the flange.

Clause 26—The device of clause 25, wherein the ridge comprises visual indicia configured to facilitate insertion of the collar to a position internal to the cervix.

Clause 27—The device of clause 26, wherein the visual indicia is a first color, and wherein the cervical portion includes a seal body including visual indicia and/or being formed from materials of a second color.

Clause 28—The device of any one of clauses 23-27, wherein the collar includes a barrier between the outer body and the cervical seal.

Clause 29—The device of clause 28, wherein the conduit further defines a first inflation port distal to the barrier, and a second inflation port proximal to the barrier.

Clause 30—A device for treating a uterus, the device including a conduit defining an inflation pathway and a second inflation pathway; a bladder coupled to the conduit and defining a first volume in fluid communication with the inflation pathway; an outer body defining a second volume; and a cervical seal coupled to the conduit and comprising a sidewall defining a third volume in fluid communication with the second inflation pathway, wherein the sidewall is of variable thickness.

Clause 31—The device of clause 30, wherein a proximal portion of the sidewall is thicker than a distal portion of the sidewall, wherein the proximal and distal portions are separated by a point of radialmost expansion of the cervical seal in the inflated state.

Clause 32—The device of clause 30 or 31, wherein the proximal portion is coupled to the conduit, and wherein the proximal portion is configured to radially flare in a proximal direction about the conduit.

Clause 33—The device of any one of clauses 30-32, wherein the bladder comprises a tip portion formed of variable thickness.

Clause 34—A device for treating a uterus, the device comprising an intrauterine portion defining a second volume and defining suction openings in communication with the second volume, wherein the intrauterine portion is formed from a resilient material having material characteristics configured to provide columnar strength for the device to be handled by or near a proximal end of the device for collapsible insertion of the intrauterine portion through a caesarian opening or a cervix and into the uterus, and further configured to return to an original shape.

Clause 35—The device of clause 34, wherein the material characteristics are further configured to provide for the intrauterine portion deforming to be contoured to a shape of the uterus from resistance from a uterine wall.

Clause 36—The device of any one of clause 34 or 35, wherein the suction openings are configured to be arranged in fluid communication with a vacuum source to draw a vacuum within the uterus to induce contraction of the uterus.

Clause 37—The device of any one of clauses 34-36, wherein the intrauterine portion is configured to be deployed to an expanded configuration within the uterus.

Clause 38—The device of clause 37, wherein the intrauterine portion further comprises an outer body, and a bladder disposed within the outer body, wherein the bladder is configured to be arranged in fluid communication with a fluid source and to receive a fluid to move the intrauterine portion to the expanded configuration.

Clause 39—The device of clause 38, wherein the second volume is defined between the outer body and the bladder.

Clause 40—The device of clause 39, wherein the bladder has a proximal end opposite a distal end, wherein the device further comprises a conduit at least partially extending through the intrauterine portion and defining a suction port that axially positioned within the outer body and proximal to the proximal end of the bladder to further define the suction path.

Clause 42—The device of clause 41, wherein the conduit further defines a distal suction port that is axially positioned within the outer body and distal to the distal end of the bladder to further define the suction path.

Clause 43—The device of any one of clauses 34-42, wherein the intrauterine portion has an expanded width in the expanded configuration is greater than an original width of the intrauterine portion, and optionally wherein the intrauterine portion comprises an extended length in the expanded configuration that is greater than an original length of the intrauterine portion.

Clause 44—A device for treating bleeding of a uterus, the device comprising an intrauterine portion configured to provide collapsible insertion of the intrauterine portion through a caesarian opening or a cervix and into the uterus, and further configured to return to an original shape, the intrauterine portion including: an outer body defining suction openings; a bladder disposed within the outer body and having a proximal end opposite a distal end; and a conduit at least partially extending through the intrauterine portion and defining a proximal suction port that is positioned within an interior of the outer body and proximal to the proximal end of the bladder.

Clause 45—The device of clause 44, wherein the conduit further defines a distal suction port that is axially positioned within the outer body and distal to the distal end of the bladder.

Clause 46—A device for treating bleeding of a uterus, the device including an intrauterine portion configured to be inserted into the uterus and comprising an outer body, and a bladder disposed within the outer body, wherein the bladder is configured to be arranged in fluid communication with a fluid source and to receive a fluid to move the intrauterine portion to an expanded configuration in which the intrauterine portion expands axially to an extended length and further expands radially to an expanded width.

Clause 47—The device of clause 46, wherein the bladder further comprises a plurality of bladders each configured to be separately inflatable to impart a desired profile to the outer body of the intrauterine portion.

Clause 48—The device of any one of clause 46 or 47, wherein the intrauterine portion defines suction openings in communication with a suction path that is defined between an inner surface of the outer body and the bladder, wherein the suction openings are configured to be arranged in fluid communication with a vacuum source to draw a vacuum within the uterus to induce contraction of the uterus.

Clause 49—The device of any one of clauses 46-48, wherein the intrauterine portion is bulbous and further comprises a head, a neck extending proximally and taping radially inwardly from the head, and a crown extending distally from the head and tapering radially inwardly to define a blunt end of the device.

Clause 50—The device any one of clauses 46-49, wherein the outer surface of the intrauterine portion defines a recess, and wherein the suction openings are disposed within the recess.

Clause 51—The device of clause 50, wherein the suction openings are equally spaced within the recess.

Clause 52—The device of clause 50 or 51, wherein the recess is oriented longitudinally along the intrauterine portion.

Clause 53—The device of any one of clauses 46-52, wherein the recess comprises a plurality of recesses, and optionally, wherein the recesses are equiangularly arranged radially about the intrauterine portion.

Clause 54—A device for treating bleeding of a uterus, the device including an intrauterine portion shaped to be deployed within the uterus and comprising an outer surface oriented radially outwardly and configured to directly contact tissue of the uterus to provide a tamponade, wherein the outer surface defines suction openings configured to be arranged in fluid communication with a vacuum source to draw a vacuum within the uterus to induce contraction of the uterus.

Clause 55—The device of clause 54, wherein the intrauterine portion defines recesses, and wherein the suction openings are disposed within the recesses.

Clause 56—The device of clause 55, wherein the suction openings are equally spaced within the recesses.

Clause 57—The device of clause 55 or 56, wherein the recesses are elongate and oriented longitudinally along the intrauterine portion.

Clause 58—The device of any one of clauses 55-57, wherein the recesses are equiangularly arranged radially about the intrauterine portion.

Clause 59—The device of any one of clauses 55-58, wherein the recesses are asymmetrically arranged radially about the intrauterine portion.

Clause 60—The device of any one of clauses 55-59, wherein the intrauterine portion is bulbous and further comprises a head, a neck extending proximally and taping radially inwardly from the head, wherein the recesses are arranged on each of the head and the neck.

Clause 61—The device of any one of clauses 55-60, wherein respective distal ends of the recesses terminate prior to a distal end of the intrauterine portion.

Clause 62—A connector for connecting two conduits each including at least three fluid pathways, the connector including: a female component comprising: an endwall; at least one fitting extending from the endwall to define a first end of the female component; an outer sidewall extending from the endwall to define a second end of the female component, wherein the outer sidewall defines a first cavity; a first inner wall coaxially disposed within the outer sidewall and extending from the endwall by a distance less than a distance by which the outer sidewall extends from the endwall, wherein the first inner wall has a first inner diameter defining a second cavity within the first cavity; and a second inner wall coaxially arranged within the first inner wall and extending from the endwall by a distance less than the distance by which the first inner wall extends from the endwall, wherein the second inner wall has a second inner diameter defining a third cavity within the second cavity.

Clause 63—The connector of clause 62, further comprising: a male component configured to be removably coupled with the female component, the male component comprising: a first section having a first outer diameter sized to be received within the first cavity of the female component, wherein the first section comprises at least one fitting and defines a first offset bore configured to be arranged in fluid communication with the first cavity of the female component; a second section coaxially arranged relative to the first section, the second section having a second outer diameter sized to be received within the second cavity of the female component, wherein the second section defines a second offset bore configured to be arranged in fluid communication with the second cavity of the female component; and a third section coaxially arranged relative to the second section, the third section having a third outer diameter sized to be received within the third cavity of the female component, wherein the second section defines a central bore arranged in fluid communication with the first cavity of the female component.

Clause 64—The coupler of clause 63, wherein the at least one fitting of the male component further comprises: a first fitting in fluid communication with the first offset bore; and a second fitting in fluid communication with the second offset bore.

Clause 65—The coupler of clause 64, wherein a face of the male component defines a non-circular opening between the first fitting and the second fitting with the non-circular opening in fluid communication with the central bore.

Clause 66—The coupler of any one of clauses 63-65, further comprising: a first seal coupled to the first section; a second seal coupled to the second section; and a third seal coupled to the third section.

Clause 67—The coupler of any one of clauses 61-64, wherein the at least one fitting of the female component further comprises: a first lateral fitting in fluid communication with the first cavity; a second lateral fitting in fluid communication with the second cavity; and a third central fitting in fluid communication with the third cavity.

Clause 68—The coupler of clause 67, wherein the third central fitting is larger than the first and second lateral fittings.

Clause 69—A system for treating a uterus, the system comprising: a device comprising a conduit defining a suction pathway, a first inflation pathway and a second inflation pathway, a bladder defining a first volume in fluid communication with the inflation pathway, an expandable outer body at least partially disposed over the bladder such that a second volume defined between the bladder and the outer body is in fluid communication the suction pathway of the conduit, and a cervical seal defining a third volume in fluid communication with the second inflation pathway; a control hub comprising: a vacuum valve configured to be arranged in fluid communication with the suction pathway of the conduit; a first inflation valve configured to be arranged in fluid communication with the first inflation pathway of the conduit; and a second inflation valve configured to be arranged in fluid communication with the second inflation pathway of the conduit.

Clause 70—The system of clause 69, further comprising: a first switch operably coupled to the vacuum valve and configured to receive a first input to actuate the vacuum valve to permit or prevent suction from a vacuum source to be drawn on the second volume; and a second switch operably coupled to the inflation valve and configured to receive a second input to actuate the inflation valve to a closed position to maintain inflation of the bladder with fluid in the first volume.

Clause 71—The system of clause 69 or 70, wherein the inflation valve comprises a self-sealing fitting configured to be coupled with a source of inflation fluid.

Clause 72—A system for treating bleeding of a uterus, the system comprising: an intrauterine portion comprising a head configured to be deployed to an expanded configuration within the uterus in which the head expands to be contoured to a shape of the uterus to provide a tamponade; a cervical portion coupled to the intrauterine portion and comprising a seal; and a controller configured to receive data or signals indicative of a state of the intrauterine portion as deployed within the uterus.

Clause 73—The system of clause 72, further comprising a sensor in communication with the sensor and configured to generate the data or signals to be transmitted to the controller, wherein the sensor is at least one of a contact sensor, a force sensor, a pressure sensor, a flow rate sensor, a light sensor, temperature sensor, and a displacement sensor configured to transmit a signal to the controller.

Clause 74—The system of clause 73, wherein the sensor is disposed on the intrauterine portion.

Clause 75—The system of any one of clauses 72-74, wherein the intrauterine portion further comprises a radiopaque marker configured to promote visualization on fluoroscopy.

Clause 76—A method of treating a uterus with a device including an intrauterine portion defining suction openings, a cervical portion coupled to the intrauterine portion, and a conduit, the method comprising the steps of: directing the intrauterine portion through a cervix or through a caesarean opening; positioning the cervical portion within the cervix; directing second inflation fluid from a second fluid source through a second inflation pathway of the conduit to within a third volume to deploy the cervical portion to an expanded configuration against the cervix; and operating a vacuum source in fluid communication with a second volume defined between a bladder and an outer body of the intrauterine portion through a suction pathway of the conduit.

Clause 77—The method of clause 76, further comprising directing second inflation fluid from a first fluid source through a first inflation pathway of the conduit to within a first volume defined by the bladder to deploy the intrauterine portion to an expanded configuration within the uterus.

Clause 78—A method of treating a uterus with a device including an intrauterine portion defining suction openings, a cervical portion coupled to the intrauterine portion, and a conduit, the method comprising the steps of: directing the intrauterine portion through a cervix or through a caesarean opening; positioning the cervical portion within the cervix; directing first inflation fluid from a first fluid source through a first inflation pathway of the conduit to within a first volume defined by the bladder to deploy the intrauterine portion to an expanded configuration within the uterus; and directing second inflation fluid from a second fluid source through a second inflation pathway of the conduit to within a third volume to deploy the cervical portion to an expanded configuration against the cervix.

Clause 79—The method of clause 78, further comprising operating a vacuum source in fluid communication with a second volume defined between a bladder and an outer body of the intrauterine portion through a suction pathway of the conduit

Clause 80—The method of any one of clauses 76-79, further comprising coupling the device to a control hub with a connector to establish fluid communication between the suction pathway and a vacuum line, the first inflation pathway and a first supply line, and the second inflation pathway and a second supply line.

Clause 81—The method of clause 80, further comprising coupling a first syringe of saline to the second supply line; actuating the first syringe of saline to direct the second inflation fluid into the second supply line to deploy the cervical portion; and decoupling the first syringe of saline, wherein the second supply line includes a self-sealing fitting.

Clause 82—The method of clause 81, further comprising coupling a second syringe of saline to the first supply line with a first inflation valve in a closed position.

Clause 83—The method of clause 82, further comprising actuating a second inflation valve at least partially to an open position; actuating the second syringe to direct the first inflation fluid through the first supply line to deploy the intrauterine portion; and further actuating the second inflation valve to the closed position, thereby maintaining the volume of fluid within the bladder and the intrauterine portion in the expanded configuration.

Clause 84—A method of treating a uterus with a device including an intrauterine portion defining suction openings, a bladder disposed within the intrauterine portion, the method comprising: inflating the bladder with fluid to expand the intrauterine portion within the uterus; providing a vacuum to within the intrauterine portion to draw blood from within the uterus through the suction openings; and deflating the bladder while maintaining the vacuum.

Clause 85—The method of clause 84, further comprising adjusting a vacuum level of the vacuum based on a magnitude of expansion of the intrauterine portion within the uterus.

Clause 86—The method of clause 85, wherein the vacuum level and/or the magnitude of expansion is selectively set according to a user input or a control protocol stored on memory of a controller.

Clause 87—The method of any one of clauses 84-86, further comprising at least partially opening a first inflation valve to selectively adjust a rate of deflation or drainage of the fluid from within the bladder based on pressure from contraction of the uterus.

Clause 88—A method of manufacturing a device for treating a uterus, wherein the device includes a conduit, a collar, a distal cap, a bladder, and an expandable outer body, the method comprising: arranging the collar to be slidable over the conduit; securing in position the collar to the conduit; positioning the distal cap to be slidable over a distal portion of the conduit; securing the bladder to the distal cap; positioning the bladder and distal cap to be slidable over a distal portion of the conduit; securing the bladder to the conduit to define a first volume; positioning the outer body over the bladder; and securing the outer body to the collar to define a second volume between the bladder and the outer body.

Clause 89—The method of clause 88, wherein the device further comprises struts, the method further comprising securing an inner surface of the outer body to the struts.

Clause 90—The method of clause 89, further comprising: positioning the struts in a jig; and overmolding the outer body on the struts.

Clause 91—The method of any one of clauses 88-90, wherein the device includes an expandable cervical seal, the method further comprising securing the cervical seal to the collar and to the conduit to define a third volume.

Clause 92—The method of any one of clauses 88-91, further comprising extruding the conduit to define lumens of different cross-sectional areas.

Clause 93—The method of clause 92, wherein a cross-sectional area of a largest of the lumens is within the range of 0.50 to 0.65 square inches.

The foregoing disclosure is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

1. A device for treating a uterus, the device comprising:
a conduit defining a suction pathway and an inflation pathway;
a bladder coupled to the conduit and defining a first volume in fluid communication with the inflation pathway; and
an expandable outer body at least partially disposed over the bladder and defining suction ports, wherein a second volume defined between the bladder and the outer body is in fluid communication with the suction ports of the outer body and the suction pathway of the conduit,
wherein the conduit defines a suction port external to the bladder and in fluid communication with the suction pathway.

2. (canceled)

3. (canceled)

4. The device of claim 1, wherein the outer body comprises spacing features configured to maintain separation between the bladder and the outer body with the bladder in an inflated state for bodily fluids to be drawn through the second volume and into the suction pathway.

5. The device of claim 4, wherein the spacing features are projections integrally formed with the outer body.

6. The device of claim 1, further comprising a strut coupled to the outer body, wherein the strut defines a channel configured to maintain separation between the bladder and the outer body with the bladder in an inflated state for bodily fluids to be drawn through the second volume and into the suction pathway.

7. The device of claim 6, wherein the strut defines bores aligned with the suction ports of the outer body, and wherein the bores are disposed within the channel.

8. The device of claim 6, wherein the strut is oriented longitudinally along an inner surface of the outer body.

9-11. (canceled)

12. The device of claim 1, wherein the bladder is formed from a resilient material configured to expand with inflation fluid being directed through the inflation pathway and into the first volume, and wherein the outer body is formed from a resilient material configured to conform to the expansion of the bladder.

13. The device of claim 1, wherein the bladder is formed from a flexible, non-resilient material configured to unfurl with inflation fluid being directed through the inflation pathway and into the first volume, and wherein the outer body is formed from a resilient material configured to conform to a shape of the bladder.

14. The device of claim 1, wherein the bladder comprises a base coupled to the conduit, and wherein the bladder is not otherwise coupled to the conduit and configured to expand omnidirectionally.

15. The device of claim 1, wherein the conduit comprises a distal end portion, wherein the device further comprises a distal cap movably disposed over the distal end portion, and wherein the bladder is coupled to the distal cap.

16. The device of claim 15, wherein the distal end portion is tapered, and wherein the distal cap comprises a blunt tip.

17. The device of claim 15, wherein the distal cap comprises a trunk, and a cap head extending distally from the trunk, wherein the cap head has an outer diameter less than an outer diameter of the trunk, and wherein a distal tip portion of the bladder is coupled to the cap head.

18. (canceled)

19. (canceled)

20. The device of claim 1, wherein the conduit defines a second inflation pathway, and wherein the device further comprises an expandable cervical seal coupled to the conduit and defining a third volume in fluid communication with the second inflation pathway.

21-28. (canceled)

29. A device for treating a uterus, the device comprising:
a conduit defining a suction pathway and an inflation pathway;
a bladder comprising a base coupled to the conduit and defining a first volume in fluid communication with the inflation pathway; and
an expandable outer body at least partially disposed over the bladder and defining suction ports, wherein a second volume defined between the bladder and the outer body is in fluid communication with the suction ports of the outer body and the suction pathway of the conduit,
wherein the conduit defines a suction port positioned proximal to the bladder, and further defines an inflation port positioned distal to the suction port and distal to the base of the bladder.

30. The device of claim 29, wherein the conduit defines another suction port positioned distal to the bladder.

31. A device for treating a uterus, the device comprising:
a conduit defining a suction pathway and a first inflation pathway, and a second inflation pathway;
a bladder coupled to the conduit and defining a first volume in fluid communication with the first inflation pathway; and
an expandable outer body at least partially disposed over the bladder and defining suction ports, wherein a second volume defined between the bladder and the outer body is in fluid communication with the suction ports of the outer body and the suction pathway of the conduit; and
an expandable cervical seal coupled to the conduit and defining a third volume in fluid communication with the second inflation pathway.

32. The device of claim 31, further comprising a collar coupled to the conduit with the outer body and fixedly coupled to the collar, wherein the collar comprises a barrier arranged to maintain fluid separation between the second volume and the third volume.

33. The device of claim 32, wherein the conduit further defines a second inflation port positioned proximal to the barrier.

34. The device of claim 32, wherein the cervical seal is formed from a sidewall comprising an outer surface opposite an inner surface to define a thickness therebetween, and wherein the thickness of the sidewall varies between opposing ends of the cervical seal.

35. The device of claim 32, wherein the suction pathway and the first and second inflation pathways are partitioned channels within the conduit, wherein the conduit has a constant outer diameter and wherein an effective cross-sectional area of the suction pathway is greater than an effective cross-sectional area of each of the first and second inflation pathways.
